SEQUENCE LISTING (7 sequences)

SEQ ID NO: 1 - DNA, 579 bp, Artificial
  lpcA gene derived from Escherichia coli MG1655
atgtaccaggatcttattcgtaacgaactgaacgaagcggcggaaacgctggctaactttttaaaagatgacgccaatattcacgccattcagcgcgcggcggtcctgttagcagacagctttaaagccggtggcaaagtgctttcctgcggcaacggcggttcccattgcgacgctatgcactttgccgaagagttgaccggtcgctaccgtgaaaaccgtccgggctacccggcgattgctatttctgacgttagtcatatttcctgcgtcggtaatgatttcggtttcaatgatattttctcccgctacgttgaagcggtaggtcgcgaaggcgatgtactgctggggatctccacctccggtaactctgcaaacgtgatcaaagcgatcgcagcggcgcgtgagaagggaatgaaagtgatcaccctgaccggtaaagacggcggcaaaatggctggcacggcggatatcgaaattcgcgtaccgcactttggttatgccgaccgcattcaggagattcacattaaagtgatccatatcctgatccagttgattgaaaaagagatggttaagtaa

SEQ ID NO: 2 - protein, 192 aa, Artificial
  LpcA protein derived from Escherichia coli MG1655
MYQDLIRNELNEAAETLANFLKDDANIHAIQRAAVLLADSFKAGGKVLSCGNGGSHCDAMHFAEELTGRYRENRPGYPAIAISDVSHISCVGNDFGFNDIFSRYVEAVGREGDVLLGISTSGNSANVIKAIAAAREKGMKVITLTGKDGGKMAGTADIEIRVPHFGYADRIQEIHIKVIHILIQLIEKEMVK

SEQ ID NO: 3 - DNA, 879 bp, Artificial
  araC gene derived from Escherichia coli MG1655
atggctgaagcgcaaaatgatcccctgctgccgggatactcgtttaacgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgcattagtcaggcgaagctgcttttgagcactacccggatgcctatcgccaccgtcggtcgcaatgttggttttgacgatcaactctatttctcgcgagtatttaaaaaatgcaccggggccagcccgagcgagtttcgtgccggttgtgaagaaaaagtgaatgatgtagccgtcaagttgtcataa

SEQ ID NO: 4 - protein, 292 aa, Artificial
  AraC protein derived from Escherichia coli MG1655
MAEAQNDPLLPGYSFNAHLVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCEEKVNDVAVKLS

SEQ ID NO: 5 - DNA, 3544 bp, Artificial
  araBAD operon derived from Escherichia coli MG1655
acccgtttttttggatggagtgaaacgatggcgattgcaattggcctcgattttggcagtgattctgtgcgagctttggcggtggactgcgctaccggtgaagagatcgccaccagcgtagagtggtatccccgttggcagaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggaaacgtgctggcgctgcgcccggagtttgccgaaaacccgaacgcgatgttcgtattgtggaaagaccacactgcggttgaagaagcggaagagattacccgtttgtgccacgcgccgggcaacgttgactactcccgctacattggtggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcctgccgccagccagtttctttgatgagctggacccgatcctcaatcgccatttgccttccccgctgttcactgacacttggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggtttggtcgcgtactcggctggccgctggaacagcttgccgcccagcatccggaactgaaaacgcaaatcaacgccagccagaaacaactgcttccggcgctgaccgaagcatgggccaaaaatccgtctctggatcacctgccggtggtgctcgactggtttaacggccgccgcacaccgaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgttaataacgtgatggcactgggcggcatcgcgcggaaaaaccaggtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagctcagcaaaaaatggccagtgcggtagagaaaaccctgcaaccgtgcagcgagcaggcacaacgctttgaacagctttatcgccgctatcagcaatgggcgatgagcgccgaacaacactatcttccaacttccgccccggcacaggctgcccaggccgttgcgactctataaggacacgataatgacgatttttgataattatgaagtgtggtttgtcattggcagccagcatctgtatggcccggaaaccctgcgtcaggtcacccaacatgccgagcacgtcgttaatgcgctgaatacggaagcgaaactgccctgcaaactggtgttgaaaccgctgggcaccacgccggatgaaatcaccgctatttgccgcgacgcgaattacgacgatcgttgcgctggtctggtggtgtggctgcacaccttctccccggccaaaatgtggatcaacggcctgaccatgctcaacaaaccgttgctgcaattccacacccagttcaacgcggcgctgccgtgggacagtatcgatatggactttatgaacctgaaccagactgcacatggcggtcgcgagttcggcttcattggcgcgcgtatgcgtcagcaacatgccgtggttaccggtcactggcaggataaacaagcccatgagcgtatcggctcctggatgcgtcaggcggtctctaaacaggatacccgtcatctgaaagtctgccgatttggcgataacatgcgtgaagtggcggtcaccgatggcgataaagttgccgcacagatcaagttcggtttctccgtcaatacctgggcggttggcgatctggtgcaggtggtgaactccatcagcgacggcgatgttaacgcgctggtcgatgagtacgaaagctgctacaccatgacgcctgccacacaaatccacggcaaaaaacgacagaacgtgctggaagcggcgcgtattgagctggggatgaagcgtttcctggaacaaggtggcttccacgcgttcaccaccacctttgaagatttgcacggtctgaaacagcttcctggtctggccgtacagcgtctgatgcagcagggttacggctttgcgggcgaaggcgactggaaaactgccgccctgcttcgcatcatgaaggtgatgtcaaccggtctgcagggcggcacctcctttatggaggactacacctatcacttcgagaaaggtaatgacctggtgctcggctcccatatgctggaagtctgcccgtcgatcgccgcagaagagaaaccgatcctcgacgttcagcatctcggtattggtggtaaggacgatcctgcccgcctgatcttcaatacccaaaccggcccagcgattgtcgccagcttgattgatctcggcgatcgttaccgtctactggttaactgcatcgacacggtgaaaacaccgcactccctgccgaaactgccggtggcgaatgcgctgtggaaagcgcaaccggatctgccaactgcttccgaagcgtggatcctcgctggtggcgcgcaccataccgtcttcagccatgcactgaacctcaacgatatgcgccaattcgccgagatgcacgacattgaaatcacggtgattgataacgacacacgcctgccagcgtttaaagacgcgctgcgctggaacgaagtgtattacgggtttcgtcgctaagtagccgcatccggtatgtaacgcctgatgcgacgctgacgcgtcttatctggcctacacgctgcgattttgtaggccggataagcaaagcgcatccggcattcaacgcctgatgcgacgctggcgcgtcttatcaggcctacgcgctgcgattttgtaggccggataagcaaagcgcatccggcattcaacgcctgatgcgacgctggcgcgtcttatcaggcctacacgctgcgattttgtaggccggataagcaaagcgcatccggcacgaaggagtcaacatgttagaagatctcaaac

SEQ ID NO: 6 - DNA, 27 bp, Artificial
  synthetic primer
aaagctcacattgttgctgtttttatc

SEQ ID NO: 7 - DNA, 28 bp, Artificial
  synthetic primer
gaagattgatttaagaattttcaggtcg

(19) United States
(12) Patent Application Publication
(10) Pub. No.: US 2013/0217132 A1
(43) Pub. Date: Aug. 22, 2013

(54) METHODS, COMPOSITIONS AND USE FOR ENHANCING CHEMICAL TOLERANCE BY MICROORGANISMS

(71) Applicants: Ryan T. Gill, Denver, CO (US); Nicholas R. Sandoval, Boulder, CO (US); Tirzah Y. Glebes, Boulder, CO (US)

(72) Inventors: Ryan T. Gill, Denver, CO (US); Nicholas R. Sandoval, Boulder, CO (US); Tirzah Y. Glebes, Boulder, CO (US)

(21) Appl. No.: 13/585,828

(22) Filed: Aug. 14, 2012

Related U.S. Application Data
(63) Continuation-in-part of application No. 12/771,845, filed on Apr. 30, 2010.
(60) Provisional application No. 61/174,940, filed on May 1, 2009.
(60) Provisional application No. 61/223,322, filed on Jul. 6, 2009.

(51) Int. Cl. C12N 15/70

(52) U.S. Cl. CPC C12N 15/70
USPC 435/471; 435/320.1; 435/252.3; 435/252.33

(57) ABSTRACT

Embodiments herein concern compositions and methods for enhancing chemical tolerance of biomass conversion by microorganisms. In some embodiments, enhancing tolerance of biomass hydrolysate conversion includes enhancing tolerance to low molecular weight organic compounds.

### CROSS REFERENCE TO RELATED APPLICATIONS

The instant application is a continuation-part-application of U.S. Non-Provisional application Ser. No. 12/771,845 filed Apr. 30, 2010, which claims the benefit under 35 U.S.C. 119(e) of U.S. Provisional Patent Application Ser. No. 61/174,940 filed on May 1, 2009 and U.S. Provisional Patent Application Ser. No. 61/223,322 filed Jul. 6, 2009. These applications are incorporated herein by reference in their entirety for all purposes.

### FEDERALLY FUNDED RESEARCH

Embodiments disclosed herein were supported in part by a grant from the U.S. Department of Energy National Renewable Energy Laboratory and Fellowship number 2007056441 from the National Science Foundation; grant BES0228584 from the National Science Foundation and an Fellowship number 2007056441 from the National Science Foundation. The U.S. government may have certain rights to practice the subject invention.

### FIELD

Embodiments herein report methods, compositions and uses for enhancing tolerance by microorganisms to toxic byproducts from biomass hydrolysis or biofuels production. This application also generally reports methods, compositions and uses of vectors or genetic manipulations to increase the production of industrial fermentation/bioprocesses. In certain embodiments, compositions and methods herein concern biomass conversion into biofuels, production of recombinant proteins for pharmaceutical application or other products generated by a microorganism. Certain embodiments relate to compositions and methods for enhancing tolerance to toxic byproducts such as low molecular weight organic compounds using methods and compositions disclosed herein. In other embodiments, intracellular levels of certain aldehydes or acetates are modulated to increase production of useful microbial byproducts. In yet other embodiments, genetic manipulations to culture of microorganism may be performed in order to increase tolerance to low molecular weight organic compounds and/or produce higher quantities of a target compound.

### BACKGROUND

Mass production of useful chemicals can produce problematic byproducts to platform organisms capable of producing these chemicals. Bacteria are capable of producing useful chemicals but often production is hindered by toxic byproducts. Escherichia coli are a well studied microorganism with a completed genome sequence commonly used in the chemical industry. However, approximately 60% of predicted genes in the genome have unknown function.

Cellulosic biomass, for example, cellulose and hemicellulose, include about 75 percent of all plant material. This material can be used as a low-grade fuel that can be burned. Currently it is difficult and costly to turn cellulosic biomass into a bio fuels such as a liquid fuel like ethanol. Cellulose and hemicellulose are polymers of sugar, but they are complex compounds not easily broken down into their simpler component sugars. Potential sources of cellulosic biomass include agricultural plant wastes, plant wastes from industrial processes (sawdust, paper pulp), and crops grown specifically for fuel production, such as switchgrass and poplar trees.

Lignocellulosic feedstocks, such as switchgrass, poplar, and corn stover, provide green house gas savings of 65-100% in comparison to petrol. Feedstocks that do not require a substantial change in land-use include crop and municipal wastes, fall grass harvests, and algae. Other potential feedstocks include waste from pulp and paper mills, construction debris, and animal manures. These feedstocks are of extreme interest because they require no additional land-use conversion.

### SUMMARY

Some embodiments herein concern modulating or conferring and/or inducing tolerance of toxic side products of biomass hydrolysis upon a microorganism. Other embodiments herein concern modulating, conferring and/or inducing genes to increase export or induce metabolism of chemical byproducts such as low molecular weight organic compounds (e.g. acetates, aldehydes for example furfurals) in a microorganism. In accordance with these embodiments, microorganisms can increase production of useful chemicals (e.g. by induction of growth, metabolism etc), other products and/or industrial fermentation, including, but not limited to biofuels from cellulosic biomass, for example, hemicellulosic and cellulosic biomass. Certain embodiments herein provide for modified microorganisms having increased chemical tolerance, or enhanced chemical export, reduced chemical import or enhanced chemical metabolism relative to its wild type and/or control type microorganism. For example, chemicals (e.g. bioproducts or toxic chemicals) contemplated herein can concern low molecular weight organic compounds including, but not limited to, formic acid, acetic acid, citric acid, propionic acid, pyruvic acid, oxalic acid, succinic acid, malonic acid, levulinic acid, formaldehyde, acetaldehyde, and butyraldehyde, furan-2-carbaldehyde (commonly known as furfural), 5-(hydroxymethyl)-2-furaldehyde (commonly known as hydroxymethylfurfural (HMF)) and phenolic aldehydes like benzaldehyde, 4-hydroxy-3-methoxybenzaldehyde (commonly known as vanillin), and (2E)-3-phenylprop-2-enal (commonly known as cinnamaldehyde) or a combination thereof. Some embodiments report use of increased low molecular weight organic compound (e.g. acetate or aldehyde) tolerance relative to wild type and/or control type organisms of use to increase production of or length of tolerance for byproduct synthesis by the organism.

In one embodiment, a modified microorganism can be a bacteria or yeast or other microorganism capable of producing products from cellulosic biomass. Other microorganism contemplated herein include, but are not limited to, Candidatus Regiella spp., Candidatus Hamiltonella, Shigella spp., Haemophilus spp., Klebsiella spp., Eikenella spp., Neisseria spp.and Francisella spp. In accordance with these embodiments, examples of species of these families include, but are not limited to Candidatus Regiella insecticola, Candidatus Hamiltonella defensa, Shigella flexneri, Haemophilus ducreyi, Klebsiella pneumonia, Eikenella corrodens, Neisseria gonorrhoeae, Neisseria meningitides, Francisella tularensis. In other embodiments, a modified microorganism can be E. coli. In yet other embodiments, a modified microorganism can be a Zymomonas spp., Saccharomyces spp., and subspecies or other bacterial species capable of producing biofuels from cellulosic biomass. In accordance with these embodiments, some modified microorganisms have an increase in tolerance or coping mechanisms for toxic byproducts of biomass conversion or industrial fermentation as demonstrated by an increase in production of these products and/or growth rate of at least about 1%-about 5%; about 1% to about 10%; about 1% to about 15%; about 1% to about 25%; to over 100% increase compared to a control microorganism population.

Other embodiments report increased copy number of specific genomic regions and chemical-resistant phenotypes of a microorganism to increase toxic chemical tolerance of that microorganism. In accordance with these embodiments, genes can include, but are not limited to, genes that code for proteins involved in construction of outer-cellular components, and genes that code for proteins which are involved in chemical-consuming pathways, chemical exporting and other genes or a combination of genes for toxic chemical inhibition, degradation, export and/or tolerance by the microorganism. Some embodiments report increased copy number of one or more genes in order to increase chemical (e.g. low molecular weight organic compounds for example, acetates, aldehydes etc.) tolerance by a bacterial organism (e.g. E. coli) or yeast. Other embodiments may use increased copy number of one or more genes or gene regions for increased chemical (e.g. low molecular weight organic compounds for example, acetates, aldehydes etc.) tolerance, chemical export, chemical degradation, and/or chemical metabolism in a bacterial organism compared to a control (e.g. pEZseq a cloning vector with no insert) that include, but are not limited to, murC (peptidoglycan biosynthesis (murein synthesis)), fumB (fumarate hydratase (TCA cycle—may consume acetate)), cadA (lysine decarboxylase (acid resistance), yjdL (predicted transporter), cadA-yjdL (two genes next to each other in the genome), argA (N-acetylglutamate synthase (acetyl-CoA consumer)), metH (methionine biosynthesis (methionine supplementation has been shown to confer acetate supplementation)), lpcA, pBTL-1 (a broad host range vector with no insert, lpcA gene in the pBTL-1 vector), nfrB, lgt-thyA operon (lpcA-pBTL1, for example the lpcA gene in the pBTL-1 vector, also known as umpA) and any combination thereof. Yet other embodiments concern genetic deletion of part or all of a gene alone or in combination to increased expression of other genes to increase tolerance to production of small organic compounds by a microorganism. Some embodiments disclosed herein concern manipulation of certain pathways to modulate tolerance to low molecular weight organic compounds from biomass hydrolysates in a microorganism including, but not limited to, formylTHF biosynthesis I, TCA cycle, glycolysis, arginine biosynthesis, peptidoglycan biosynthesis, lysine biosynthesis, methionine biosynthesis and combinations thereof.

Other vectors contemplated of use in some embodiments herein include, but are not limited to, pEZseq (Lucigen) cloning systems, pSMART cloning systems (Lucigen), pACYCDuet cloning systems (Novagen) pBMT-1, pBMT-2, pBMT-3, pBMT-4, pBMT-5, pBMT-6, pBT-1, pBT-2, pBT-3, pBT-4, pBT-5, pBT-6, pBMTB-1, pBMTB-2, pBMTB-3, pBMTB-4, pBMTB-5, pBMTB-6, pBTB-1, pBTB-2, pBTB-3, pBTB-4, pBTB-5, pBTB-6, pBMTL-1, pBMTL-2, pBMTL-3, pBMTL-4, pBMTL-5, pBMTL-6, pBTL-1, pBTL-2, pBTL-3, pBTL-4, pBTL-5, pBTL-6 or any vector capable of inducing the one or more genes.

In accordance with these embodiments, one or more gene(s) may be increased in expression or copy number to modulate chemical tolerance in a bacterial organism or a combination of genes or gene segments may be used. In yet other embodiments, other genes may be deleted or modified to modulate chemical tolerance in a bacterial organism.

In certain embodiments, compositions of use to modulate tolerance to low molecular weight organic compounds found in biomass hydrolysate may include one, two, three, or four, or up to all genes disclosed herein alone or in combination with other compositions to modulate tolerance to these compounds. In certain embodiments, modulation of genes in bacterial organisms may include modulation of low molecular weight organic compounds tolerance such as acetate tolerance by a bacterial organism. In accordance with these embodiments, one or more gene(s) may be modulated in a bacterial organism to modulate acetate metabolism including, but not limited to, lpcA, murC, fumB, cadA, yjdL, argA, metH, and any combination thereof.

In other embodiments, a culture of microorganism can be manipulated in order to increase production of, or tolerance for ethanol, hydrolosates, acetate or furfural by methods and compositions disclosed herein. Manipulations of these organisms can include, but is not limited to, deletions, insertions, increase in gene copy number, and induction of genes, in order to increase production of, or tolerance for ethanol, hydrolysates, acetate or furfural production. Some embodiments include assessing the organism's ability to produce lipopolysaccharide (LPS). Selection of an organism capable of producing increased levels of LPS compared to another organism can indicate that the organism is better suited to tolerate production of ethanol, hydrolysates, acetate or furfural. Therefore, LPS production levels can be a marker for increased tolerance or production of low molecular weight organic compounds. In accordance with these embodiments, a microorganism can be E. coli or other similar bacterial organism.

In certain embodiments, one strain of a microorganism may be better suited for production or tolerance of ethanol, hydrolysates, acetate or furfural than other strains. For example, a strain that produces higher levels of LPS or other similar molecule may be more suited to tolerate increased concentrations of a target molecule. In accordance with these embodiments, an E. coli culture comprising higher LPS production is contemplated of use to produce target molecules disclosed herein. In certain E. coli strains compositions and methods disclosed herein can be used to manipulate a strain into having lpcA overexpression or manipulating other genes important in lipopolysaccharide (LPS) core biosynthesis in order to increase production of LPS or to generate strain populations capable of producing LPS to the level of a control organism. Strains that may be of use to tolerate higher concentration levels of ethanol, hydrolysates, acetate or furfural compared to a control can be BW25113 or a manipulated MG1665 strain of E. coli. In certain embodiments, a bacterial strain may contain an araC and/or araBAD deletion or an araB deletion alone or in combination with overexpression of lpcA.

In other embodiments, modulation of tolerance for low molecular weight organic compounds found in biomass hydrolysates in bacterial organisms may include modulation of one or more pathways or genes associated with these pathways.

In other embodiments, one or more gene(s) may be modulated in a bacterial organism to modulate tolerance of low molecular weight organic compounds having a terminal carbonyl group (e.g. aldehyde), including, but not limited to, lpcA, lgt, nfrB, thyA or combinations thereof or other genes in combination with these genes in order to modulate low molecular weight organic compounds having a terminal carbonyl group production in biomass hydrolysate by a microrganism. In certain embodiments, compositions and methods disclosed herein concern modulating tolerance in a microorganism to furfural and/or hydroxymethylfurfural.

Low molecular weight organic compounds of biomass hydrolysates contemplated in some embodiments disclosed herein can include, but are not limited to, formic acid, acetic acid, citric acid, propionic acid, pyruvic acid, oxalic acid, succinic acid, malonic acid, levulinic acid or any combination thereof. Aldehydes contemplated herein can include, but are not limited to, compounds composed of organic molecules with 10 or less carbons and a formyl group side chain. Carbons of these aldehydes can be oriented in straight-chain conformations or cyclic orientations with one or many side chains. Examples of straight-chain aldehydes include, but are not limited to, formaldehyde, acetaldehyde, and butyraldehyde and combinations thereof. Examples of cyclic aldehydes include, but are not limited to, furan-2-carbaldehyde (commonly known as furfural), 5-(hydroxymethyl)-2-furaldehyde (commonly known as hydroxymethylfurfural) and phenolic aldehydes like benzaldehyde, 4-hydroxy-3-methoxybenzaldehyde (commonly known as vanillin), and (2E)-3-phenylprop-2-enal (commonly known as cinnamaldehyde) and combinations thereof. Modulation of tolerance by microorganisms to any combination of compounds disclosed herein is contemplated.

Some embodiments disclosed herein can include modifying microorganisms to express any genes disclosed herein within the organism and/or cloning or stably integrating additional copies or a predetermined copy number of the disclosed genes into a microorganism for modulating tolerance to a low molecular weight organic compound. Compositions contemplated herein to modulate tolerance to low molecular weight organic compounds may include amino acid supplements.

### DESCRIPTION OF ILLUSTRATIVE EMBODIMENTS

### Definitions

As disclosed herein “low molecular weight organic compounds” can mean low molecular weight organic acids or carbonyl-containing compounds having ten carbons or less. In certain embodiments, these compounds can be linear or cyclic and can include, but are not limited to, formic acid, acetic acid, citric acid, propionic acid, pyruvic acid, oxalic acid, succinic acid, malonic acid, levulinic acid, formaldehyde, acetaldehyde, butyraldehyde, furan-2-carbaldehyde (commonly known as furfural), 5-(hydroxymethyl)-2-furaldehyde (commonly known as hydroxymethylfurfural) and phenolic aldehydes like benzaldehyde, 4-hydroxy-3-methoxybenzaldehyde (commonly known as vanillin), and (2E)-3-phenylprop-2-enal (commonly known as cinnamaldehyde) and combinations thereof.

As disclosed herein “modulate” can mean an increase, a decrease, upregulation, downregulation, an induction or the like.

### BRIEF DESCRIPTION OF THE FIGURES

The following drawings form part of the present specification and are included to further demonstrate certain embodiments of the present invention. The embodiments may be better understood by reference to one or more of these drawings in combination with the detailed description of specific embodiments presented herein.

FIG. 1 represents a schematic of a cellulosic biomass conversion to biofuels.

FIGS. 2A and 2B represent histograms representing clone growth in the presence of A) 1.75 g/L selection or B) 2.5 g/L selection media.

FIGS. 3A and 3B illustrate clone growth under various conditions.

FIG. 4 illustrates bacterial growth under various conditions in the presence or absence of various amino acids.

FIG. 5 illustrates a schematic of hydrolysate inhibitors and their attack on a bacteria.

FIG. 6 represents a blown up region representing a clone fitness mapped over an E. coli genome.

FIGS. 7A-7B represents a schematic of toxic byproduct effects in a microorganism and data collected.

FIGS. 8A-8D represents a blow-up region representing a clone fitness mapped over an E. coli genome and data collected.

FIGS. 9A-9D represents a blow-up region representing a clone fitness mapped over an E. coli genome and data collected.

FIG. 10 represents a histogram plot of certain clones and predominate genes contributing to growth of the organism under certain conditions.

FIG. 11 represents a histogram plot of data obtained under various growth conditions of a microorganism in the presence or absence of targeted gene induction.

FIG. 12 represents a table of various target pathways of some embodiments disclosed herein.

FIG. 13 represents a plot of clone fitness mapped over a bacterial genome, peak size is relative to fitness.

FIG. 14 represents a plot of clone fitness mapped over a bacterial genome, peak size is relative to fitness.

FIG. 15 represents a schematic of gene linkage contemplated regarding some embodiments disclosed herein.

FIG. 16 represents tolerance profile differences between certain microorganisms in biomass hydrolysates at 24 and 48 hours.

FIGS. 17A-17C illustrate identification of genes (A) by certain methods disclosed herein; comparison of tolerance to certain agents in the presence and absence of a target tolerance construct (B); and (C) a histogram plot comparing different E. coli strains and in the presence of various concentrations of a toxic byproduct from biomass hydrolysis.

FIGS. 18A and 18B illustrates an exemplary metabolic pathway (A) that include target genes of various embodiments disclosed herein and (B) a histogram plot illustrating tolerance by microorganism of a toxic byproduct from biomass hydrolysis with/or without overexpression of a target gene of some embodiments herein.

FIG. 19 represents a histogram plot of growth of certain clones under certain conditions.

FIG. 20 represents a schematic of gene linkage in certain clones.

FIG. 21 represents a schematic representing a blown up region of a genome in a microorganism contemplated herein in some embodiments.

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that concentrations, times, temperature and other details may be modified through routine experimentation. In some cases, well known methods or components have not been included in the description.

In accordance with embodiments of the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Creating biofuels from biomass is an important tool for moving away from dependence on depleting sources of fuels. In addition, enhancing industrial fermentation and/or bioprocessing is needed for increased production and reduced cost. In certain embodiments herein, using cellulosic biomass for generating biofuels in microorganisms is contemplated. Examples of cellulosic biomass include, but are not limited to, using hemicellulosic, lignocellulosic and cellulosic biomass for generating biofuels.

Biofuels derived from lignocellulosic biomass hold promise for making up a significant fraction of this market. One goal is pretreatment to relax the crystalline nature of cellulose for downstream hydrolysis, convert hemicellulose to pentoses, and to remove lignin. Harsh conditions used in pretreatment create a variety of toxic compounds that inhibit the fermentation performance. General classes of inhibitors have been defined: acetic acid release from actylxylan decomposition, furan derivatives from sugar and subsequent degradation, and phenolic compounds derived from lignin. Furan derivatives include, for example, furfural (2-furaldehyde) and 5-hydroxymethylfurfural (HMF), which result from pentose and hexose degradation, respectively. Subsequent degradation of these compounds introduces formic acid (furfural and HMF degradation) and levulinic acid (HMF degradation) into the hydrolysate. Phenolic compounds include, for example, acids, alcohols, aldehydes, and ketones.

Although many fermentative microorganisms exist, in certain embodiments, Escherichia coli, Saccharomyces cerevisiae, and Zymomonas mobilis can serve as industrial biocatalysts for biofuels production. Each microorganism has limitations in native substrate utilization, production capacity, or tolerance. Unlike Saccharomyces cerevisiae or Z. mobilis, E. coli natively ferments both hexose and pentose sugars. Ethanologenic E. coli also has higher tolerance to lignocellulosic inhibitors than its fermentative counterparts.

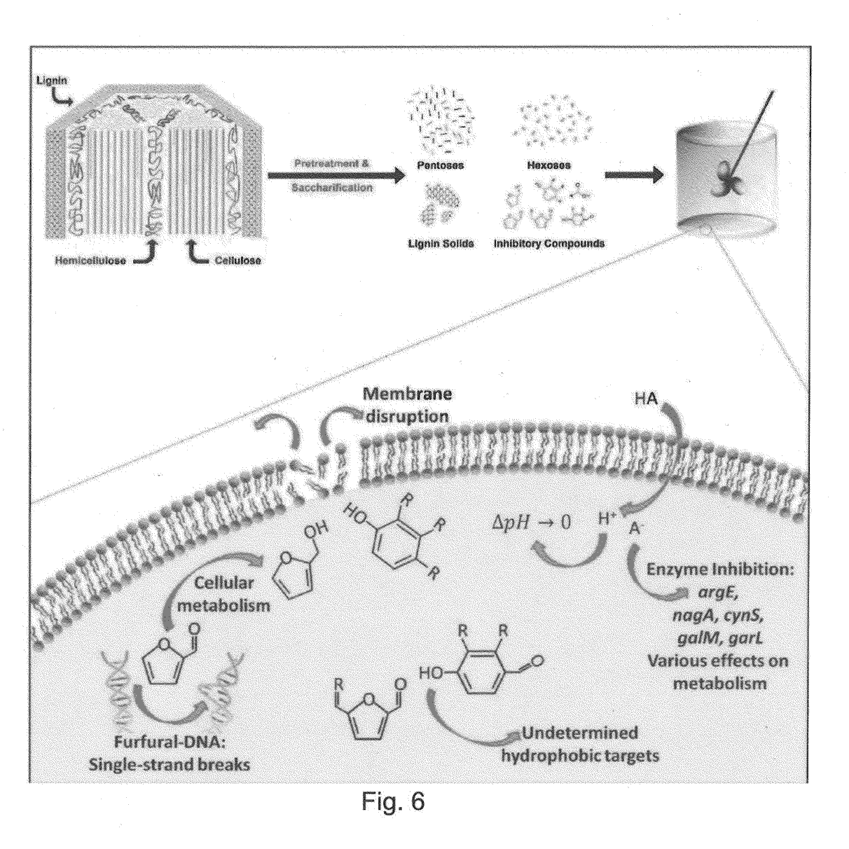

As depicted in FIG. 6, generally accepted categories of antimicrobial activity for inhibitors in lignocellulosic hydrolysate include: (a) compromising the cell membrane, (b) inhibiting essential enzymes, or (c) negative interaction with DNA or RNA. These toxic compounds often act by inhibiting multiple targets. Although efforts are underway to limit the amount and types of inhibitors created during pretreatment, at the present time, economically viable processes still fall short. Regardless of pretreatment optimization, inhibitors such as acetic acid, released directly from hemicelluloses decomposition, remain in the hydrolysate. Thus, a need to engineer more tolerant fermentative microorganisms exists.

### Organic Acids

Organic acids derived from lignocellulosic biomass pretreatment and subsequent saccharification inhibit the growth and metabolism of microorganisms (e.g. E. coli). This, in turn, reduces the yield, titer, and productivity of biofuel fermentation. Various organic acids are created in pretreatment steps: acetic acid is derived from the hydrolysis of acetylxylan, a main component of hemicellulose; others (formic, levulinic, etc.) are a result from degraded sugars.

Acetic acid is usually found at the highest concentration in the hydrolysate. Levels of acetate depend on the type of cellulosic biomass and the pretreatment method. Concentrations can range from 1 to >10 g/L in the hydrolysate. Formic acid, while more toxic to for example, E. coli than acetic acid, is typically present at concentrations much less than that of acetic acid (commonly 10% of acetic acid concentrations). Other toxic weak acids, whose hydrolysate concentrations are rarely reported, are present at an even lower concentration than formic acid.

### Some Modes of Toxicity

Weak organic acids have been shown to primarily inhibit the production of cell mass, but not the fermentation itself. Acetate is a natural fermentation product that is known to accumulate due to “overflow metabolism” and inhibit cell growth. Acetate concentrations as low as 0.5 g/L have been shown to inhibit cell growth by 50% in minimal media.

Weak acids, in the undissociated form can permeate the cell membrane, and once inside, dissociate to release the anion and the proton. These “uncoupling agents” can disrupt the transmembrane pH potential since, effectively, a proton is allowed across the membrane without the creation of ATP. This dissociation of the weak acid inside the cytoplasm is due to the fact the intracellular pH, pHi, is naturally at a pH of ˜7.8, which is much higher than the weak acid's pKa. As these acids dissociate inside the cell, the pHi decreases, which can inhibit growth. External pH has a large affect on the toxicity of the weak acids. E. coli KO11 in LB media with 5.0 g/L acetate reached an ethanol titer twice as fast at an initial pH of 7.0 compared to initial pH of 6.0, and thrice as fast compared to an initial pH of 5.5. When E. coli LY01 was subjected, at a starting pH of 6.0, to acetic, formic, or levulinic acid at the IC50 obtained at a neutral pH, the growth rate decreased to 0, 35, and 10 percent that of control growth. Formic acid may be more toxic due to the fact it has an extraordinarily high permeability through the membrane. This external pH effect is, in part, due to the fact that the acid exists in its undissociated form at higher concentrations, allowing for higher permeation of the cell membrane.

The anion also has an inhibitory effect. The anion accumulates inside the cell, which can affect the cell turgor pressure. Inhibition has been shown to be anion specific. When E. coli inhibition from acetate was compared to benzoate, the same growth rate was observed for differing pHi (7.26 for benzoate and 7.48 for acetate). It has been reported that the toxicity of weak acids depended highly on the hydrophobicity of the acid.

### Furan Derivatives

Furan derivatives are a result of sugar degradation during pretreatment. Furfural and HMF are the primary derivatives appearing in lignocellulosic hydrolysate. Concentrations typically range between 0-6 g/L for each compound. As previously mentioned, levulinic and formic acid are also formed via degradation of these aldehydes. While dilute acid hydrolysis is a common method for pretreatment, acidic conditions are known to cause degradation of a small fraction of the sugar monomers. Hemicellulose is the second most abundant renewable polysaccharide, averaging 25-35% of viable lignocellulosic biomass composition. Therefore, processes that avoid degradation of the C5 and C6 monomers may be important.

Aldehydes are known to have detrimental effects in microorganisms. For example, it was shown that formaldehyde denatures and interacts with polynucleotides. Formaldehyde is also known to cause protein-protein cross-linking. Methylgloxal, a dicarbonyl compound, has been shown to inhibit E. coli growth and protein synthesis at concentrations of 0.07 g/L.

### Some Modes of Toxicity

Furfural has been identified as a key inhibitor in lignocellulosic hydrolysate because it not only is toxic by itself but is also known to act synergistically with other toxins. Hydrophobicity is a marker of an organic compound's toxicity. Highly hydrophobic compounds have been shown to compromise membrane integrity. Intracellular sites are more likely to be the primary inhibition targets of furfural and HMF. In contrast, both 2-furoic acid and furfuryl alcohol have been shown to cause significant membrane leakage. Furfuryl alcohol is less toxic on a concentration basis, by almost an order of magnitude (˜2 g/L for furfural vs. ˜20 g/L furfuryl alcohol) than furfural.

Ethanol production was inhibited in E. coli LYO1 by furfural, suggesting a direct effect on glycotic and/or fermentative enzymes. In the same study, furfural inhibition on aldehyde dehydrogenase (A1DH; EC 1.2.1.5) and the pyruvate dehydrogenase (PDH) complex were investigated and determined to be in this case, more significant than ADH, as evidenced by more than 80% activity reduction in the presence of 0.12 g/L furfural, whereas ADH activity was only inhibited by 60%. This study suggests that furfural may detrimentally affect multiple glycotic enzymes that contribute to central metabolism.

Furfural and HMF have shown cytotoxic characteristics towards both bacteria and yeast. Furfural is a known dietary mutagen and has been under investigation for direct effects on DNA in the past. A series of studies confirmed that furfural-DNA interactions occur. Furfural-treated double-stranded DNA led to single-strand breaks after undergoing in vitro incubation with furfural, primarily at sequence sites with three or more adenine or thymine bases. Later, plasmids treated with furfural were observed to cause either an increase (high furfural concentrations) or decrease (low furfural concentrations) in plasmid size via insertions, duplications, or deletions.

### Phenolic Compounds

Hydrolysates can contain up to 30% lignin content for a variety of feedstocks. Major phenolic compounds have carboxyl, formyl, or hydroxyl group functionalities and arise from degradation of lignin during pretreatment. Ketones can also be released during pretreatment, but are not generally considered as primary inhibitors because they occur at low concentrations (<0.05 g/L) and are also partially or completely removed with various detoxification treatments. Most of the lignin and its derivatives are insoluble; after dilute acid pretreatment of yellow poplar, no more than 15% of the total lignin feedstock content was converted to a soluble species. Concentrations of aromatic monomers after dilute acid washes have been measured between 0-3 g/L and include acids, alcohols, and aldehydes. Due to the number of lignin-derived compounds needing to be analyzed, sequential studies with E. coli have been limited. As such, only the most commonly studied compounds are reviewed in this work.

### Modes of Toxicity

A series of studies comparing aldehydes, acids, and alcohols appearing in hydrolysate were performed with the ethanologenic E. coli LYO1. In general, the degree of toxicity correlated with the compound's octanol/water partition coefficient, log(Poctanol/water), which is a measure of hydrophobicity. In all studies the phenolics were more toxic than aliphatics or furans with the same functional group. This observation that hydrophobicity was related to membrane damage was only true for the alcohols tested, with the exception of hydroquinone. Aromatic acids caused partial membrane leakage while the aromatic aldehydes caused no significant membrane damage. A synergistic binary combination was observed for guaiacol and methylcatechol, but a less than additive combination was observed for vanillyl alcohol and all lignin-derived alcohols tested (catechol, coniferyl, guaiacol, hydroquinone, and methylcatechol). Vanillin, which is a phenolic aldehyde, was found to be a bacteriostatic and membrane active inhibitor, causing partial disruption of K+ gradients in E. coli MC1022. This finding is similar to the effect of methylglyoxal on E. coli. Membrane destabilization was experienced by 29% of the population after treatment with vanillin for one hour at over three times the minimum inhibitory concentration, but restored to 13% when grown overnight. In addition, this study showed that ATP production continues without significant interruption. In previous reports, membrane damage was found to not contribute significantly to toxicity. From these data, hypotheses have been developed stating that other cellular hydrophobic components may be the primary target for inhibition.

### Modes of Tolerance

From the studies conducted on E. coli LYO1, tolerance to aldehydes benefited from increased inoculum size, suggesting metabolism of the compound. For example, recombinant E. coli are capable of converting aromatic aldehydes to their corresponding acids. Non-lignin derived aromatic acids have also been showed to be metabolized as sole carbon sources, similar to observations of furfural and HMF metabolism. Conversion of an aldehyde to carboxylic acid or alcohol is often beneficial for E. coli due to the reduced toxicity of the functional group.

### Engineering Tolerance

Genomic library selection is a powerful tool that can discover genes or operons that, with increased copy number, confer a desired phenotype. The advent of DNA microarrays has made it easier to identify these beneficial genes. SCALEs (Scalar Analysis of Library Enrichments), and its predecessor PGTM (Parallel Gene Trait Mapping), have used E. coli genomic library selection and microarrays to engineer tolerance to Pine-Sol antibiotic, antimetabolites, 3-hydroxypropionic acid, and naphthol. Genomic selections employing libraries of heterologous genes have also been used to engineer tolerance.

Biofuels production must find cost effective and sustainable feedstocks. The commercial potential of bio fuels largely depends on the abundance and cost of the feedstock. As this number grows, commercial processes will necessarily rely more heavily upon lignocellulosic biomass. Much work is still required to improve the efficiency of fermentations of biomass hydrolysate to levels cost competitive with fermentation of pure sugar streams. Emphasis should be placed upon not only further reducing the cost of enzymatic hydrolysis step but also upon better understanding of hydro lysate toxicity mechanisms and methods for engineering tolerance. More specifically, elucidating the modes of action of specific compounds present in hydrolysate may prove critical since the levels of inhibition of various aldehydes and weak acids can vary greatly.

In accordance with these embodiments, increasing tolerance of microorganisms to toxic byproducts of biomass hydrolysis and other processes are disclosed herein. In certain embodiments, increasing growth rates of microorganisms by increasing tolerance to toxic byproducts can be accomplished by genetic manipulation of pathways, for example by modulating one or more genes intricate to the pathway. Some embodiments concern modulating tolerance to toxic byproducts by genetic manipulation of pathways that induce chemical tolerance. In other embodiments, increased tolerance of microorganisms to toxic chemicals can include increased production of recombinant proteins by microorganisms for pharmaceutical applications.

Sustainable production of biofuels will require the efficient utilization of lignocellulosic biomass. One barrier involves the creation of growth-inhibitory compounds chemical pretreatment steps, which ultimately reduce the efficiency of fermentative microbial biocatalysts. The primary toxins include organic acids, furan derivatives, and phenolic compounds. Furan derivatives, which result from degradation of lignocellulosic sugars, have been shown to hinder fermentative enzyme function. Phenolic compounds, formed from lignin, can disrupt membranes and are hypothesized to interfere with the function of intracellular hydrophobic targets.

Aldehydes can be inhibitory compounds to microorganisms, formed during the pretreatment of for example, lignocellulosic biomass. Furan derivatives, such as furfural and hydroxymethylfufural are sugar degradation products, whereas phenolic aldehydes, such as benzylaldehyde and vanillin, are monomers released from lignin. These compounds reduce the efficiency of microbial biocatalysts in biofuel production, recombinant molecule production and biorefining applications.

Acetates can be inhibitory compounds to microorganisms, for example acetates can disturb cellular homeostasis, lower pH, create an accumulation of intracellular anion, retard growth at concentrations as low as 0.5 g/L, reduce cellular glutamate and aspartate pools, as well as other interfering functions. Certain embodiments disclosed herein concern compositions and methods for modulating these and other acetate interferences to microorganisms.

Certain embodiments herein concern modulating one or more pathways capable increasing low molecular weight organic compound tolerance in a microorganism. For example, other embodiments concern modulation of one or more pathways including, but not limited to, formylTHF biosynthesis I, glycolysis, arginine biosynthesis, peptidoglycan biosynthesis, lysine biosynthesis, methionine biosynthesis or combinations thereof.

In certain embodiments, modulation of one or more genes can include modulation of lpcA, a gene capable of modulating ADP-L-glycero-b-D-manno-heptose biosynthesis to increase acetate tolerance of a microorganism. In accordance with these embodiments, a microorganism may have increased tolerance to low molecular weight organic compounds less than ten carbons (e.g acetates and/or aldehydes) having modulated expression or copy number in the microorganism. In other embodiments, modulation of one or more genes can include modulation of cadA capable of modulating one or more of POLYAMSYN pathway, aminopropylcadaverine biosynthesis, and/or lysine degradation to modulate acetate tolerance of a microorganism. Modulation of tolerance to acetates can include increasing a microorganism's tolerance for acetate for example, inducing metabolism, reducing importation or excretion of acetates from the microorganism. In certain embodiments, modulation of one or more genes can include, but are not limited to, modulation of lpcA, murC, fumB, cadA, yjdL, argA, metH or a combination thereof to increase low molecular weight organic compound tolerance of a microorganism.

In some embodiments, modulation of a gene can include modulation of lpcA (e.g. for aldehydes and acids tolerance induction), lgt (e.g for aldehyde tolerance), nfrB (e.g. for aldehyde tolerance), and thyA (e.g. for aldehyde tolerance). Two of these genes, lgt and thyA, naturally occur within the E. coli chromosome as an operon, or a single transcriptional unit and may be induced simultanously. Three of these aldehyde tolerance genes relate to membrane-localized modifications or proteins. In certain embodiments, one or more of these membrane localized modifications may be targeted to modulate tolerance in a microorganism to low molecular weight organic compounds of biomass hydrolysis. For example, gene lpcA is the first committed step in lipopolysaccharide (LPS) core biosynthesis. In certain embodiments, the substrate in which it catalyzes an isomerization reaction is D-sedoheptulose-7-phosphate. The second membrane-related gene, lgt, is involved in prolipoprotein modification through its protein product, lgt, which is an essential membrane protein. The third gene, nfrB, is an inner membrane subunit that has been attributed to N4 bacteriophage adsorption. thyA, is involved directly in de novo DNA synthesis. In addition, this DNA synthesis step is coupled with the process of producing folate derivatives within the cell. Folate derivatives are used for multiple reactions within the cell, but one specific derivative, tetrahydrofolate, THF, is also associated with a gene found during the acid selection, metH. This gene is found associated with formylTHF biosynthesis I as is meth, discussed previously.

Given the potential for using E. coli for cellulosic biofuels production, a genome-scale, gene-to-trait mapping method, SCALEs (Lynch et al. 2007, incorporated herein by reference in its entirety) to identify genes for which increased copy number increases E. coli tolerance to some inhibitors in lignocellulosic hydrolysate (e.g. furfural and acetate), as well as ethanol, a biofuel product. SCALEs has previously been applied to a broad range of application. Growth selection in the presence of furfural was performed and the SCALEs method was used to map tolerance genes, and compared to data previously obtained for other molecules in order to identify genes that broadly conferred tolerance to furfural, acetate and ethanol. This analysis implicated the genetic region encompassing the lpcA open reading frame (ORF) in tolerance to all three compounds, thus representing a potential general mechanism for conferring biofuels-relevant tolerance. The lpcA gene encodes the sedoheptulose 7-phosphate (S7P) isomerase enzyme, which catalyzes the first committed step in lipopolysaccharide (LPS) core heptose component production. Overexpression of the lpcA ORF was sufficient to confer increased tolerance to furfural, acetate, ethanol, and to increase LPS levels in E. coli.

Then, upon transferring the overexpression plasmid to another K-12 derivative, E. coli MG1655, the increased tolerance phenotype was lost, as MG1655 was natively more tolerant. It was demonstrated that BW25113 is LPS-deficient relative to MG1655. This deficiency correlates with decreased tolerance to furfural, acetate, and ethanol. Deletion of the araC regulatory gene or mutation of the function could partially restore LPS and tolerance in BW25113 to furfural, acetate, and ethanol. These results hold important implications for studies employing BW25113 and derivatives of the Keio Collection (previously described) as a host for basic discovery as well as strain design and construction.

Some embodiments contemplated herein concern lpcA overexpression in a microorganism to increase tolerance to low molecular weight organic compound production in the microorganism. In certain embodiments, overexpression of lpcA in a bacterial culture can increase tolerance to ethanol, hydrolysates, furfural and/or acetate production. Some embodiments concern. E. coli production of such compounds when lpcA is overexpressed or in the presence of increased copy number of lpcA genes to increase tolerance to ethanol, furfural and/or acetate production by the E. coli. In other embodiments, bacterial organisms that produce increased levels of LPS can be isolated and cultured for use in production of target molecules disclosed herein. Other embodiments concern manipulating the LPS pathway by deletion or insertion of genes to increase production of LPS for improving bacterial tolerance or production of target molecules such as ethanol, furfural and acetate or hydrolysates. In accordance with these embodiments, it is contemplated that araC and/or araBAD can be deleted or mutated in a bacterial organism to increase LPS production and induce tolerance and/or production. lpcA overexpression can also be used alone or in combination to confer tolerance to the target molecules.

Some embodiments disclosed herein provide for organisms and methods enhanced production and lowered toxicity of ethanol, furfural and acetate in the presence of an increased amount of cellular LPS. As disclosed herein, high production of ethanol, furfural and acetate is correlated with increased production of LPS compared to a control. When expression of araC and/or araBAD in the organism is reduced or eliminated, for example by deletion of all or a portion of araC and/or araBAD as illustrated in the Examples, LPS production is increased and the toxicity of ethanol, furfural and acetate is reduced. In addition, lpcA overexpression is known to induce tolerance of these same molecules and is the first committed step in LPS production.

Other embodiments report increased copy number of specific genomic regions and chemical-resistant (e.g. aldehyde-resistant, acetate-resistant) phenotypes. In accordance with these embodiments, genes can include, but are not limited to, genes that code for proteins involved in construction of outer-cellular components, and genes that code for proteins which are involved in toxic chemical-consuming pathways and other genes or a combination of genes for toxic chemical inhibition and tolerance. Some embodiments report increased copy number of one or more genes in order to increase chemical tolerance by an organism (e.g. acetate, aldehyde, in a bacterial organism). Other embodiments may use increased copy number of one or more genes or gene regions compared to a control (e.g. pEZseq a cloning vector with no insert) that include, but are not limited to, murC (peptidoglycan biosynthesis (murein synthesis)), fumB (fumarate hydratase (TCA cycle—may consume acetate)), cadA (lysine decarboxylase (acid resistance), yjdL (predicted transporter), cadA-yjdL (two genes next to each other in the genome), argA (N-acetylglutamate synthase (acetyl-CoA consumer)), metH (methionine biosynthesis (methionine supplementation has been shown to confer acetate supplementation)), acs (acetyl-CoA synthetase (acetate consumer)), insl (operon, ins(N, I, O)-1-DNA recombination), lpcA, pBTL-1 (a broad host range vector with no insert), and lpcA-pBTL1 (the lpcA gene in the pBTL-1 vector) and a combination thereof. In accordance with these embodiments, one gene may be increased in expression or copy number to increase acetate tolerance of a bacterial organism or a combination of genes or gene segments may be used. Yet other embodiments report modulation of one or more genes or genetic regions to increase acetate tolerance of a bacterial organism including, but not limited to, lpcA, murC, fumB, cadA, yjdL, argA, metH, lgt-thyA operon, thyA, nfrB or combinations of two or more thereof.

Vectors contemplated of use in some embodiments herein can include, but are not limited to, pEZseq (Lucigen) cloning systems, pSMART cloning systems (Lucigen), pACYCDuet cloning systems (Novagen) pBMT-1, pBMT-2, pBMT-3, pBMT-4, pBMT-5, pBMT-6, pBT-1, pBT-2, pBT-3, pBT-4, pBT-5, pBT-6, pBMTB-1, pBMTB-2, pBMTB-3, pBMTB-4, pBMTB-5, pBMTB-6, pBTB-1, pBTB-2, pBTB-3, pBTB-4, pBTB-5, pBTB-6, pBMTL-1, pBMTL-2, pBMTL-3, pBMTL-4, pBMTL-5, pBMTL-6, pBTL-1, pBTL-2, pBTL-3, pBTL-4, pBTL-5, pBTL-6 or any vector capable of inducing the one or more genes.

Some embodiments concern modulating tolerance to toxic byproducts by genetic manipulation of pathways that induce aldehyde tolerance. In other embodiments, modulating tolerance of microorganisms to toxic aldehydes may be accomplished by increasing export of, metabolism of, or hardiness of a microorganism to toxic aldehydes. In accordance with these embodiments, a microorganism having at least one of these traits may be used for example, to produce altered amounts of recombinant proteins for pharmaceutical applications, biofuels or other products from microorganisms. In certain embodiments, modulation of one or more genes can include modulation of lpcA, lgt, nfrB, thyA or a combination thereof (e.g. for modulation of aldehyde tolerance).

Certain embodiments herein concern modulating export of toxic aldehydes by a microorganism to maintain predetermined detrimental levels of low molecular weight organic compounds (e.g. acetates, aldehydes). For example, some embodiments herein report maintaining an intracellular level approximately in equilibrium with extracellular concentrations, about 0.1 to about 5 g/L of furfural by the microorganism. Other embodiments concern modulation of metabolism of toxic aldehydes in a microorganism to increase toxic aldehyde tolerance of the microorganism.

Yet other embodiments concern modulation of importing toxic aldehydes by the microorganism. For example, genetic manipulation of the microorganism to reduce uptake of toxic low molecular weight organic compounds is contemplated. In one embodiment, a genome-wide plasmid-based library selection was performed on solid minimal media supplemented with furfural or other toxic aldehyde to identify genetic elements that confer increased tolerance to furfural (see for example, Table 1). In other embodiments, modulated doubling times and cell densities can be observed for clones expressing one or more of these genes exposed to toxic aldehydes (e.g. furfural solutions), compared to a wild-type control.

lgt-thyA operon lgt has been shown to be involved in the lipid modification of prolipoprotein by transferring the sn-1,2-diacylglyceryl group from phosphatidylglycerol to the sulfhydryl group of the N-terminal cysteine. Sequence analysis, mutation, and expression studies show that lgt and the gene immediately downstream, thyA, form an operon. Mutation, complimentation, and membrane separation experiments show that Lgt is an essential membrane protein. Sequence analysis, chemical inactivation, mutation, and complementation studies indicate that His-103 is essential for the activity of the enzyme, and Tyr 235 and His-196 also have significant roles in its function. thyA is involved in DNA synthesis, namely thymidylate synthase, conversion of dUMP to dTMP is the main pathway of de novo dTMP synthesis in the cell. In addition, this DNA synthesis step is coupled with the process of producing folate derivatives within the cell. nfrB is an inner membrane subunit, associated with bacteriophage adsorption.

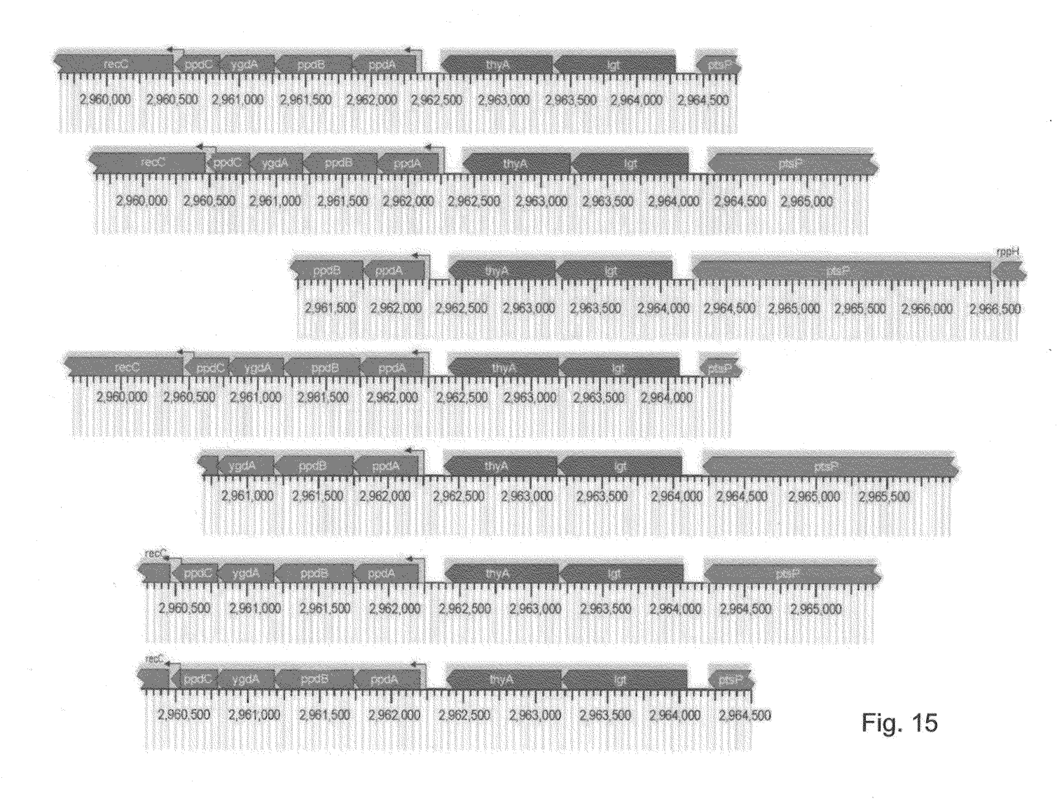

Modulation as disclosed herein may mean inducing or inhibiting, for example, expression or activity of one or more of genes or gene clusters outlined (see for example FIG. 15) above and/or up-regulating or down-regulating the expression of a genetic component identified herein to increase or decrease toxic aldehyde tolerance of a microorganism.

Certain embodiments concern biorefining, biomass (crops, trees, grasses, crop residues, forest residues, etc) and using biological conversion, fermentation, chemical conversion and catalysis to generate and use compounds. These compounds can then subsequently be converted to valuable derivative chemicals. However, of low molecular weight organic compounds may be toxic by nature and thus inhibitory to the production organisms at relatively low levels. In order to optimize production, engineering tolerance to the organic acid may be a factor. This can be accomplished by supplying exogenous molecules to enhance tolerance or to inhibit expression of a non-permissive molecule thereby permitting increased levels of conversion. Since commodity chemicals exist in a competitive environment, optimization might be necessary for the economic feasibility of processing biomass into biofuels or industrial. Therefore, compositions and methods disclosed herein are directed toward identifying bacterial strains and genetic regions within molecules that increase tolerance to toxic aldehydes for use in bioproduction products and systems.

In various embodiments, growth can be enhanced by identifying genes that with increased or decreased expression can increase the tolerance to toxic of low molecular weight organic compounds. Genetic screens, used to detect individual compounds, often proceed one cell at a time. Selections are tied to viability in a specific environment. Therefore, in one embodiment, bacterial organisms that demonstrate increased growth or tolerance for toxic aldehydes may be selected for and the genetic region that affects growth, production and/or tolerance identified. In one embodiment, modulation of genes disclosed herein demonstrate increased growth or tolerance for toxic of low molecular weight organic compound production.

Certain embodiments concern a gene region that is capable of enhancing tolerance of toxic aldehyde or acetate production and/or increasing production of biomass conversion of a microorganism. In accordance with these embodiments, expression of certain molecules within particular genomic regions may be capable of tolerance of production of toxic aldehydes. For example, strains already engineered to convert biomass such as cellulosic biomass can be modified using genetic engineering technologies disclosed herein to a) increase the conversion of biomass and/or b) increase tolerance of the strain to toxic of low molecular weight organic compounds. In addition, these methods may be used in conjunction with the SCALEs technology (Provisional Application No. 60/611,377 filed Sep. 20, 2004 and U.S. patent application Ser. No. 11/231,018 filed Sep. 20, 2005, both entitled:” Mixed-Library Parallel Gene Mapping Quantitation Microarray Technique for Genome Wide Identification of Trait Conferring Genes” incorporated herein by reference in their entirety), for genetic alterations of organisms and for genetic selection strategies.

Genetic manipulation of microorganisms can be used to make desired genetic changes that can result in desired phenotypes and can be accomplished through numerous techniques including but not limited to using a i) vector to introduce new genetic material, ii) genetic insertion, disruption or removal of existing genetic material, as well as, iii) mutation of genetic material or any combinations of i,ii, and iii, that results in desired genetic changes with desired phenotypic changes. A vector can be defined as any genetic element used to introduce new genetic material into an organism and can include, but is not limited to, a plasmid of any copy number, an intergratable element that integrate at any copy into the genome, a virus, phage or phagemid. Genetic insertions, disruptions or removals can be defined as inserting a new genetic element into the genome, disruption transcription or normal regulatory function via insertion that can affect larger regions of the genome in addition to those at the site of insertion, and the deletion or removal of a region of the genome. These can be done with techniques including, but not limited to, directed knock outs or mutations, gene replacements, transposons, random mutagenesis or a combination thereof. Mutations can be directed or random, utilizing any techniques requiring vectors, insertions, disruptions or removals, in addition to those including, but not limited to, error prone or directed mutagenesis through PCR, mutator strains, and random mutagenesis.

Analysis of Library Enrichment (SCALEs), a new high-resolution, genome-wide approach that can be used to monitor enrichment and dilution of individual clones within a genomic-library population, was recently developed. This method includes creation of representative genomic libraries with varying insert size, growth of clones in selective environments, interrogation of the selected population using microarrays, and a mathematical multi-scale analysis to identify the gene(s) for which increased copy number improves overall fitness. In certain embodiments, selections were performed on solid media supplemented with an aldehyde (furfural). Surviving colonies were used to inoculate clones cultures, from which plasmid DNA was extracted and sequenced to reveal the library insert sequence.

The SCALEs method may be employed to develop the technique of directed strain selection for relevant toxic aldehyde and acetate tolerance phenotypes. Initial selections carried out in continuous culture (e.g. E. coli, Zymomonas spp. and subspecies) with different concentrations of toxic of low molecular weight organic compounds revealed various tolerant phenotypes. In certain methods, growth rates were increased in various cultures reflecting an increase in toxic of low molecular weight organic compounds tolerance due to one or more of modulation of transporters, amino acid production and various energy production.

Genomic libraries are a common methods for performing plasmid-based overexpression selections. Individual clones conferring a desirable phenotype (e.g., of low molecular weight organic compound tolerance) within a genomic-library population can be selected for using genomic libraries. This method includes creation of representative genomic libraries with varying insert size, growth of clones in selective environments, and sequencing surviving cells.

Organisms contemplated of use herein include but are not limited to any bacterial culture capable of producing a product, sensitive to increased production of toxic of low molecular weight organic compounds, for example Escherichia coli, Pseudomonas putida, Psedumonas aeruginosa, Zymomonas spp. and subspecies (e.g. Zymomonas mobilis), Clostridia acetobutylicum, Clostridia beijerinckii, Sacchoromyces cerevisiae, Pichia pastoris or combinations thereof are contemplated.

Traditional methods to engineer cells has relied upon multiple rounds of random mutation and selection of those cells that show improved traits. In addition to being laborious these methods cause mutations that are largely ineffective as well as produce cells that appear “sick”. Traditional methods also fail to identify those mutations that are beneficial toward conferring the cellular trait. To address these limitations, pools of synthetic DNA containing molecular barcode tags, regulatory elements and gene homology regions that allow precise insertion upstream of ˜4000 genes in E. coli. The pool of synthetic DNA is then transformed into E. coli and chromosomal insertion is catalyzed by the bacteriophage λ-Red proteins, termed “Recombineering”. Insertion of synthetic regulatory elements increase or decrease downstream gene expression. Insertion of barcode tags allows genome-wide identification of mutants in a complex population on a universal microarray. Beneficial mutations can be accumulated by successive rounds of selection and insertion. Multiplex DNA synthesis and multiplex recombineering are currently being optimized.

### Nucleic Acids

In various embodiments, isolated nucleic acids may be used as test compounds for increasing toxic of low molecular weight organic compounds tolerance in a microorganism. The isolated nucleic acid may be derived from genomic RNA or complementary DNA (cDNA). In other embodiments, isolated nucleic acids, such as chemically or enzymatically synthesized DNA, may be of use for capture probes, primers and/or labeled detection oligonucleotides.

A “nucleic acid” includes single-stranded and double-stranded molecules, as well as DNA, RNA, chemically modified nucleic acids and nucleic acid analogs. It is contemplated that a nucleic acid may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000 or greater nucleotide residues in length, up to a full length protein encoding or regulatory genetic element.

### Construction of Nucleic Acids

Isolated nucleic acids may be made by any method known in the art, for example using standard recombinant methods, synthetic techniques, or combinations thereof. In some embodiments, the nucleic acids may be cloned, amplified, or otherwise constructed.

The nucleic acids may conveniently comprise sequences in addition to a portion of a lysine riboswitch. For example, a multi-cloning site comprising one or more endonuclease restriction sites may be added. A nucleic acid may be attached to a vector, adapter, or linker for cloning of a nucleic acid. Additional sequences may be added to such cloning and sequences to optimize their function, to aid in isolation of the nucleic acid, or to improve the introduction of the nucleic acid into a cell. Use of cloning vectors, expression vectors, adapters, and linkers is well known in the art.

### Recombinant Methods for Constructing Nucleic Acids

Isolated nucleic acids may be obtained from bacterial or other sources using any number of cloning methodologies known in the art. In some embodiments, oligonucleotide probes which selectively hybridize, under stringent conditions, to the nucleic acids of a bacterial organism. Methods for construction of nucleic acid libraries are known and any such known methods may be used.

### Nucleic Acid Screening and Isolation

Bacterial RNA or cDNA may be screened for the presence of an identified genetic element of interest using a probe based upon one or more sequences. Various degrees of stringency of hybridization may be employed in the assay. As the conditions for hybridization become more stringent, there must be a greater degree of complementarity between the probe and the target for duplex formation to occur. The degree of stringency may be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium. In certain embodiments, the degree of complementarity can optimally be about 100 percent; but in other embodiments, sequence variations in the RNA may result in <100% complementarity, <90% complimentarity probes, <80% complimentarity probes, <70% complimentarity probes or lower depending upon the conditions. In certain examples, primers may be compensated for by reducing the stringency of the hybridization and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. Other exemplary conditions are disclosed in the following Examples. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids may be completely complementary to a target sequence or may exhibit one or more mismatches.

### Nucleic Acid Amplification

Nucleic acids of interest may also be amplified using a variety of known amplification techniques. For instance, polymerase chain reaction (PCR) technology may be used to amplify target sequences directly from bacterial RNA or cDNA. PCR and other in vitro amplification methods may also be useful, for example, to clone nucleic acid sequences, to make nucleic acids to use as probes for detecting the presence of a target nucleic acid in samples, for nucleic acid sequencing, or for other purposes.

### Synthetic Methods for Constructing Nucleic Acids

Isolated nucleic acids may be prepared by direct chemical synthesis by methods such as the phosphotriester method, or using an automated synthesizer. Chemical synthesis generally produces a single stranded oligonucleotide. This may be converted into double stranded DNA by hybridization with a complementary sequence or by polymerization with a DNA polymerase using the single strand as a template. While chemical synthesis of DNA is best employed for sequences of about 100 bases or less, longer sequences may be obtained by the ligation of shorter sequences.

### Kits

In certain embodiments, a kit contemplated herein may include means for supplying a microorganism with increased ability to tolerate toxic chemical (e.g. of low molecular weight organic compounds, acetates, aldehydes) production and/or modulate byproduct production. Contemplated herein are means for modulating one or more genes or gene clusters capable of increasing toxic chemical tolerance of a microorganism. Some embodiments report kits having one or more compositions for increasing copy number of genes or gene regions of bacterial (e.g. E. coli) cultures disclosed herein that increase low molecular weight organic compounds tolerance of the bacterial culture. Other kits may include compositions having one or more gene or gene regions for transfecting a bacterial culture to increase acetate tolerance of the bacterial culture. The kits may include a container means. Any of the kits will generally include at least one vial, test tube, flask, bottle, syringe or other container means, into which the testing agent, may be preferably and/or suitably aliquoted. Kits herein may also include a means for comparing the results such as a suitable control sample such as a positive and/or negative control. In yet other embodiments, kits may include one or more vector for inducing one or more genes selected from the group consisting of lpcA, lgt, nfrB, thyA, murC, fumB, cadA, yjdL, argA, metH and a combination thereof.

### EXAMPLES

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

### Example 1

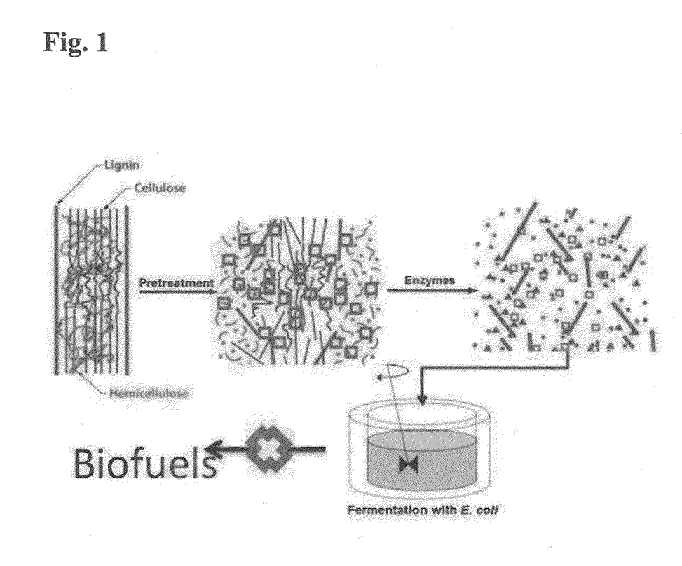

FIG. 1 represents a schematic of a cellulosic biomass conversion to biofuels.

Hydrolysate inhibitors. Lignocellulosic biomass is processed into component sugars, lignin solids, and inhibitory compounds. These inhibitors can affect microbial growth in various ways, including DNA mutation, membrane disruption, intracellular pH drop, and other cellular targets.

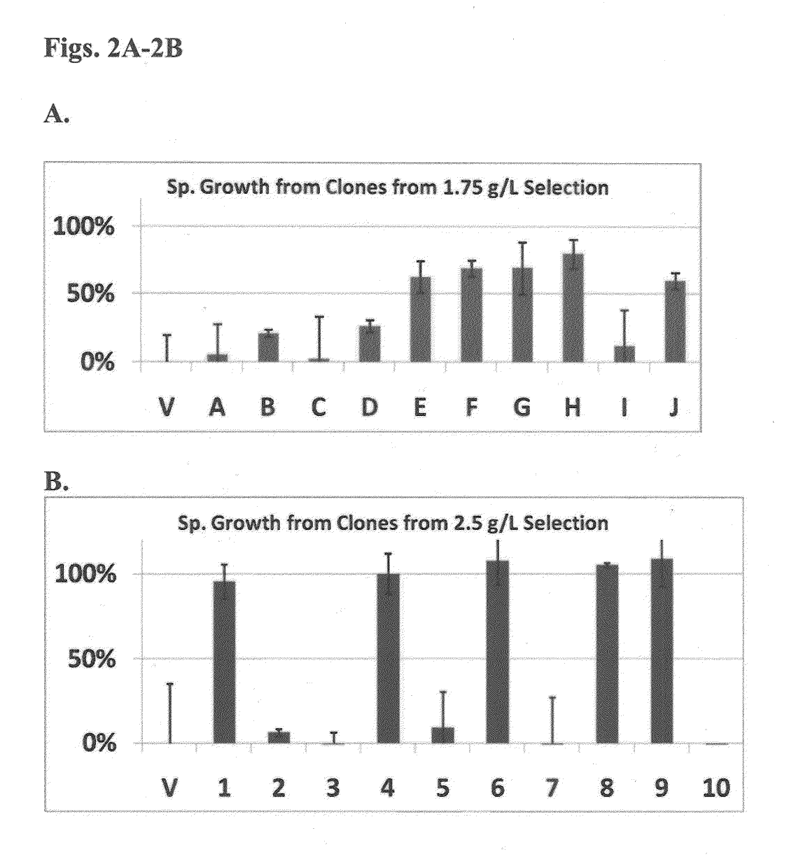

Confirmation of clones found after selection. In one example, after selection, 10 clones were randomly picked from LB Agar+Carb100 plates. All clones plus control were tested for specific growth rate at the specified concentration. Specific growth rate was plotted as percent increase or decrease compared to control specific growth. In one exemplary method bacterial cultures were exposed to two different concentrations of acetate and clones were selected for increased growth in the presence of the increased acetate. These exemplary methods are represented in FIGS. 2A and 2B. FIGS. 2A and 2B represent histograms of clone growth in the presence of A) Clones E, F, G, H, and J in the 1.75 g/L study contained the lpcA gene, and B) Clones 1, 4, 6, 8, and 9 in the 2.5 g/L study contained the lpcA gene. One method used herein was the Scales method previously described.

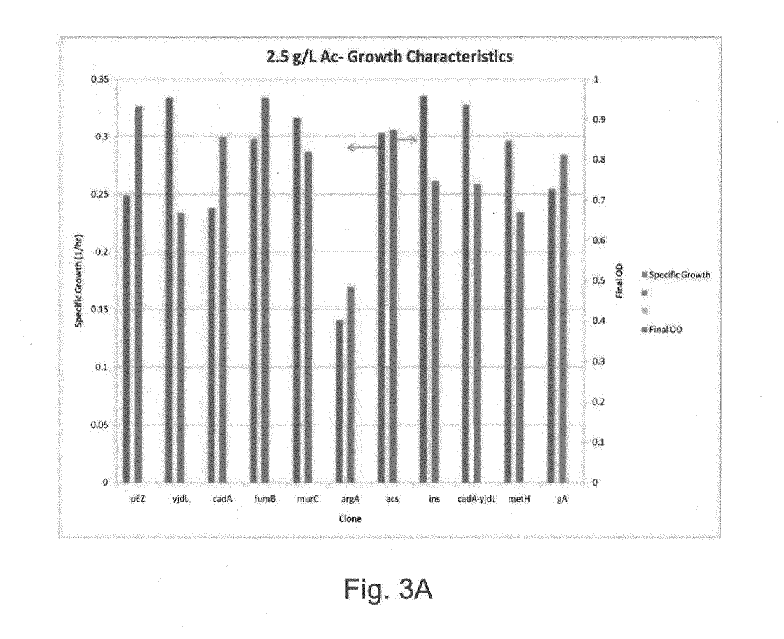

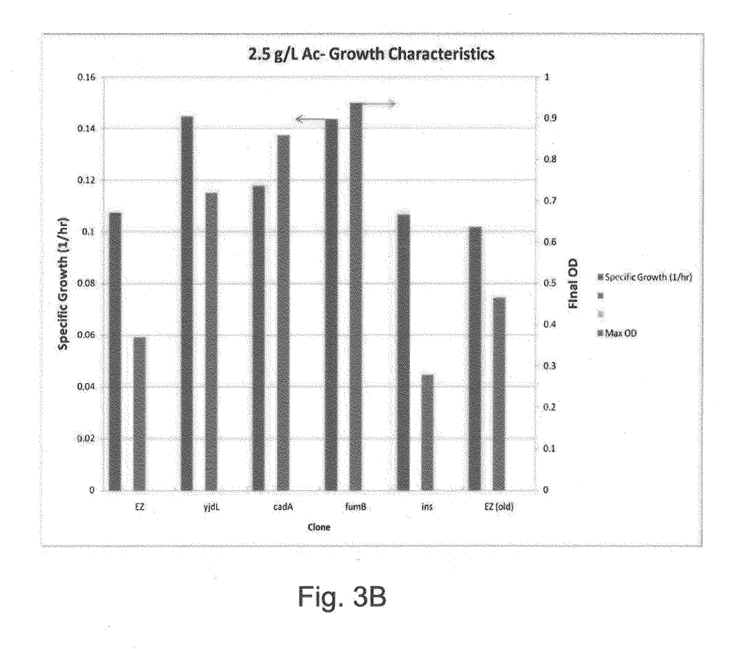

FIGS. 3A and 3B Confirmation of Clone Growth. Overnight cultures were prepared from freezer stocks. Stationary phase overnight cultures were used for a 2.5% inoculation of 5 mL MOPS minimal medium plus carbenicillin. The OD600 was monitored until the culture reached an OD600=0.2. Growth curves were constructed by introducing a 5% inoculation into 5 mL MOPS minimal medium plus carbenicillin supplemented with prepared acetic acid solution to a final concentration 2.5 g/L in a 15 mL centrifuge tube or with 50 mL of media in a 250 mL shake flask. Stock acetic acid solution was prepared by titrating 5 mL of an HPLC-grade 50% acetic acid solution (Fluka) on ice with 10 M KOH to neutral pH. Cultures were incubated at 37° C. and were shaken at 225 rpm. OD600 was monitored over the course of exponential growth and final measurements were taken after 24 hours. Specific growth rate was calculated by linear regression on the natural logarithm of the exponential phase OD600 over time. For each clone, 3A and 3B, the left bar depicts the specific growth of the clone (1/hr). The right bar depicts the final OD of the clone (proportional to the highest population achieved).

### Example 2

### Method for Growth Rate Testing

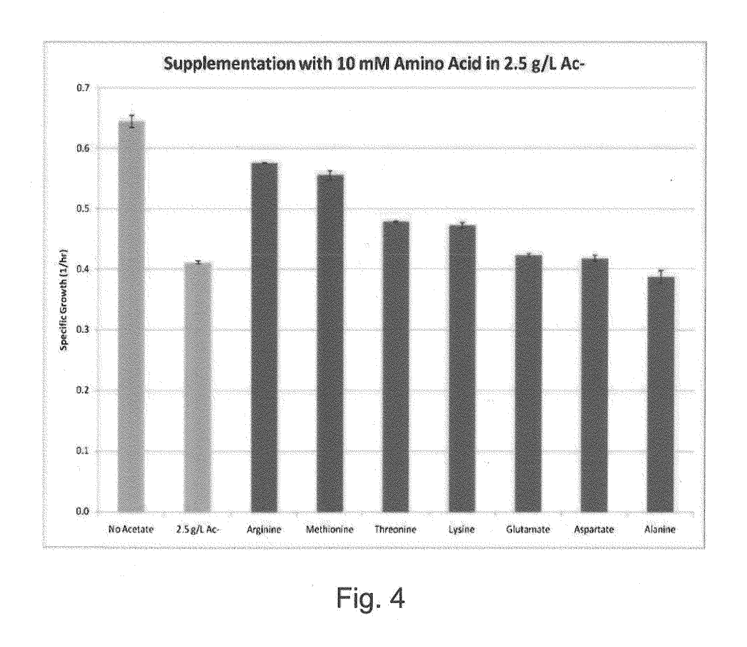

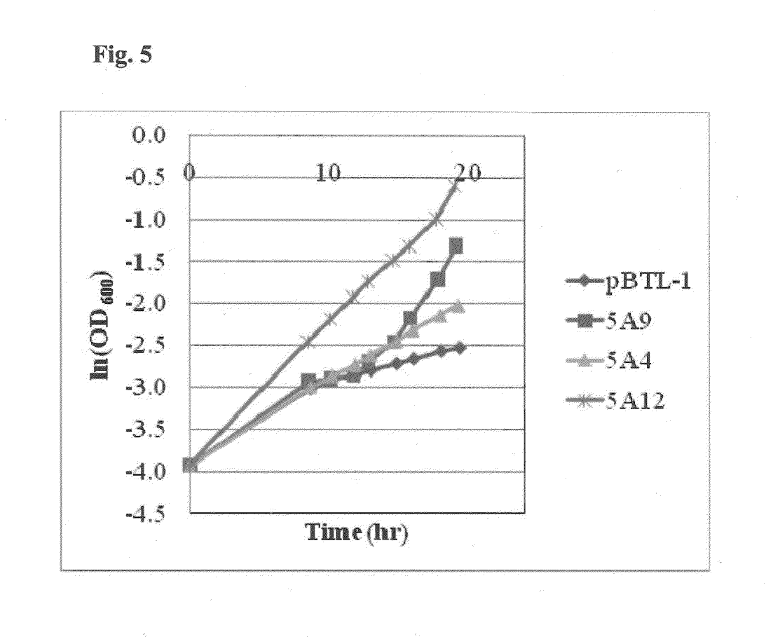

FIG. 4. In another exemplary method, amino acid supplementation of cultures was observed and illustrated by histogram plot. Overnight cultures were prepared from freezer stocks. Stationary phase overnight cultures were used for a 2.5% inoculation of 5 mL MOPS minimal medium plus carbenicillin. The OD600 was monitored until the culture reached an OD600=0.2. Growth curves were constructed by introducing a 5% inoculation into 5 mL MOPS minimal medium plus carbenicillin supplemented with prepared acetic acid solution to a final concentration 2.5 g/L in a 15 mL centrifuge tube or with 50 mL of media in a 250 mL shake flask. Stock acetic acid solution was prepared by titrating 5 mL of an HPLC-grade 50% acetic acid solution (Fluka) on ice with 10M KOH to neutral pH. Amino acid supplementation was done by preparing stock solutions of amino acids and supplementing the media to a final concentration of 10 mM. Cultures were incubated at 37° C. and were shaken at 225 rpm. OD600 was monitored over the course of exponential growth and final measurements were taken after 24 hours. Specific growth rate was calculated by linear regression on the natural logarithm of the exponential phase OD600 over time. Supplementation of arginine and methionine give the greatest increase in growth rate.

### Example 3

Confirmation of pilot SCALEs selection on furfural tolerance by log-transformation of growth curve data. Clone isolates from the pilot SCALEs furfural selection were grown in 5 ml MOPS minimal media, 100 μg carbenicillin/ml, and 1 g furfural/1 supplemented in 15 ml conical tubes at 37° C. The optical density of 1 ml culture was measured with a spectrophotometer at 600 nm. A natural log transformation of the data were then calculated and are shown here in order to observe relevant lag times and specific growth. The blank vector, pBTL-1, served as the control. Clone 5A4 contains the operon lgt-thyA, Clone 5A9 contains the gene lpcA, and Clone 5A12 contains the gene nfrB. It should be noted that these clones are direct isolates from the SCALEs pilot selection and therefore are products of library construction (e.g., fragments or whole genes are also included within the vector's insert DNA). These clones serve as the parent clones from which genes of interest could be subcloned.

This data supports that there are multiple genes that can be attributed to conferring furfural tolerance. Both Clones 5A12 (nfrB parent clone) and 5A4 (lgt-thyA parent clone) represent cultures that do not undergo the characteristic lag phase experienced by pBTL-1 control cells. Clone 5A9 (lpcA parent clone) undergoes a lag phase, but then in followed by a significantly increased specific growth after about 12 hours.

### Example 4

Hydrolysate inhibitors. Lignocellulosic biomass is processed into component sugars, lignin solids, and inhibitory compounds. These inhibitors can affect microbial growth in various ways, including DNA mutation, membrane disruption, intracellular pH drop, and other cellular targets. FIG. 6 represents a schematic of some examples of sites of low molecular weight organic compound attack on a microorganism.

### Example 5

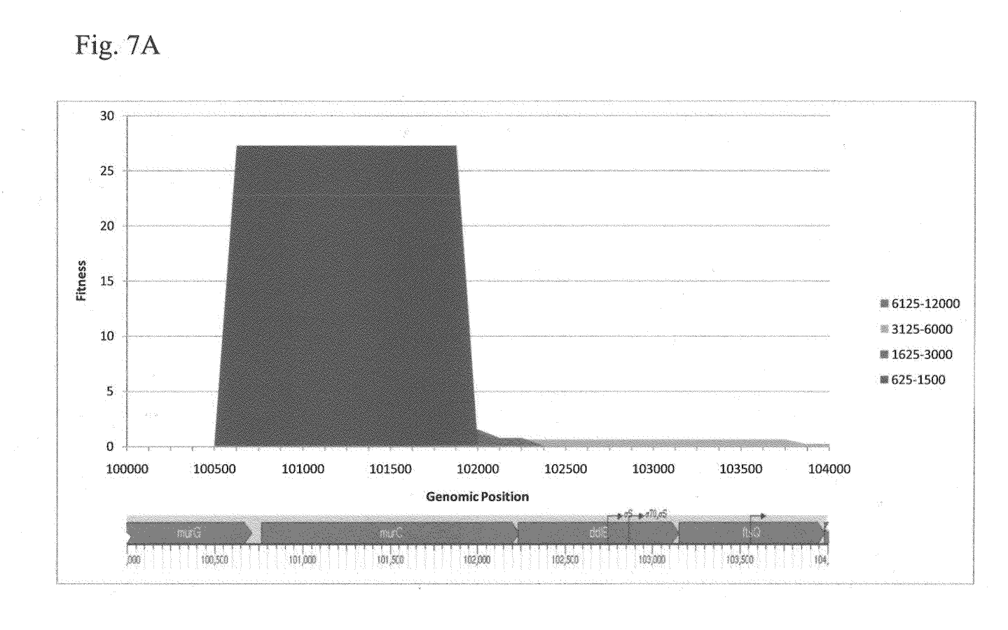

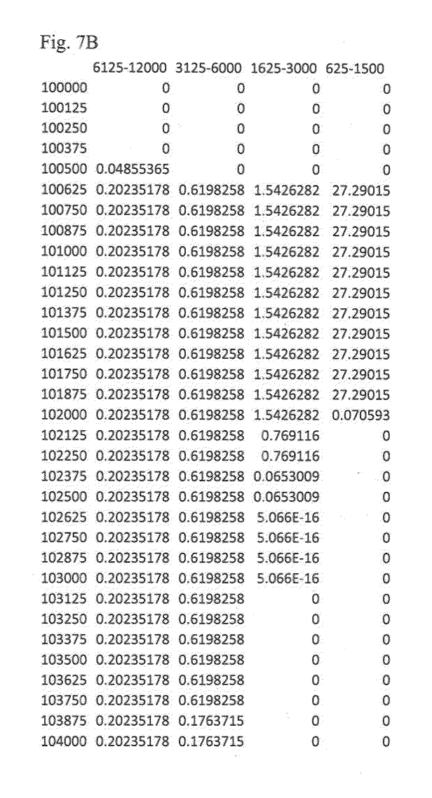

In some exemplary methods, blow-up regions are represented in FIGS. 7A and 7B. Clone fitness mapped over E. coli genome. Peak size is relative to fitness. Colors denote the size of the clones: Blue 1 kb, Red 2 kb, Green 4 kb, and Purple 8 kb (shown in black and white). In detail, fitness value plotted over genomic position. Here is illustrated gene murC and surrounding area from the 1.75 g/L SCALEs selection and analysis.

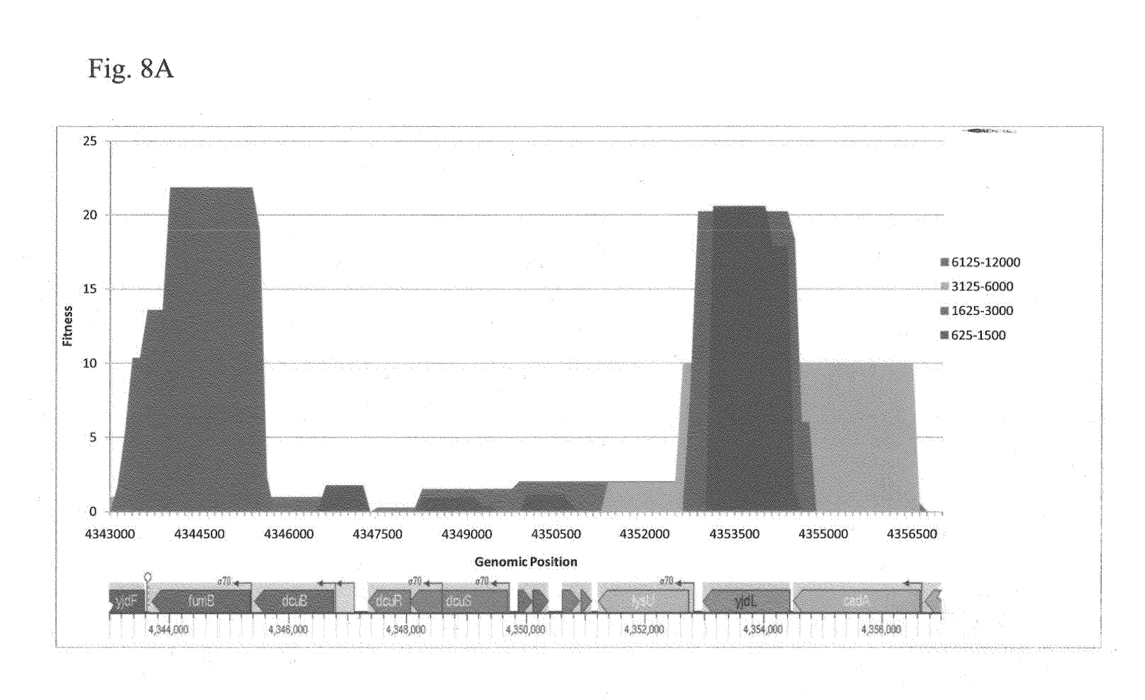

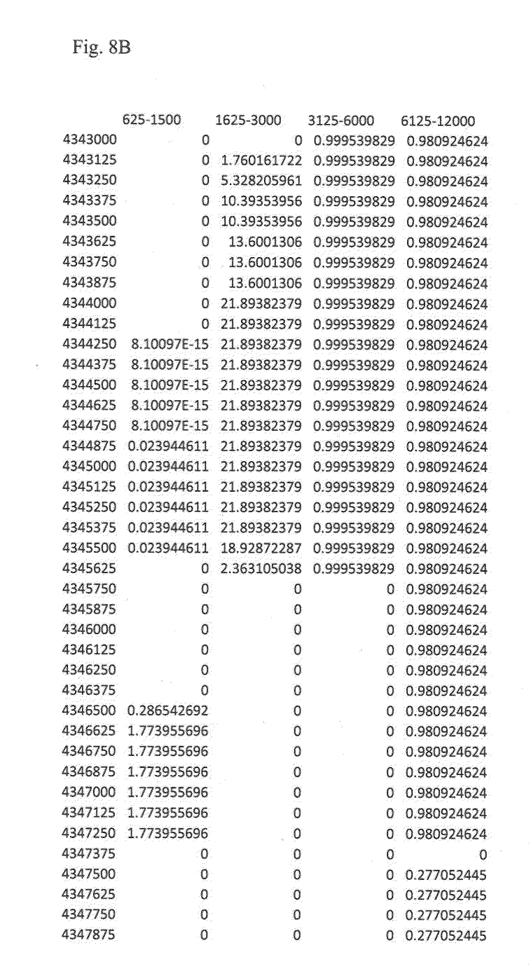

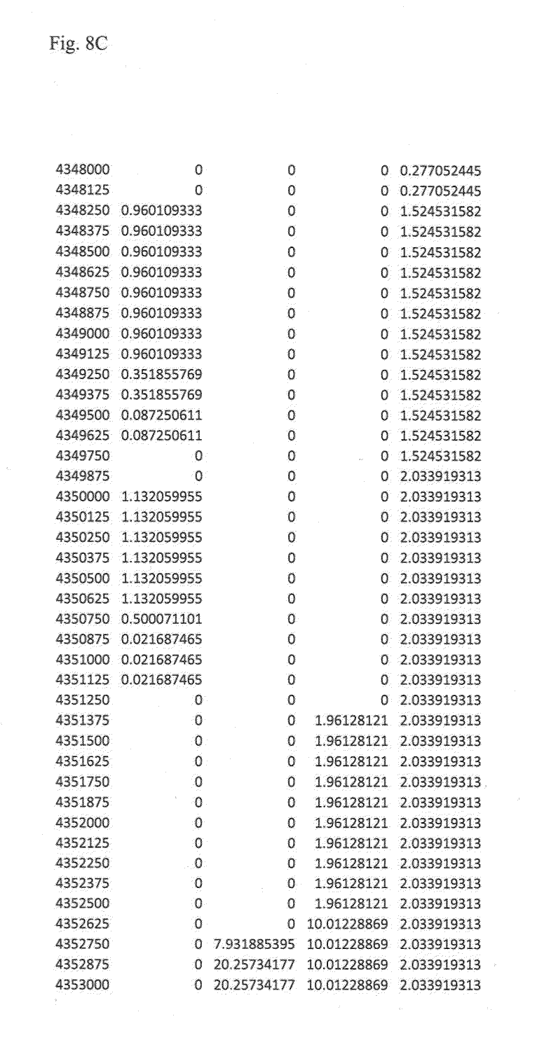

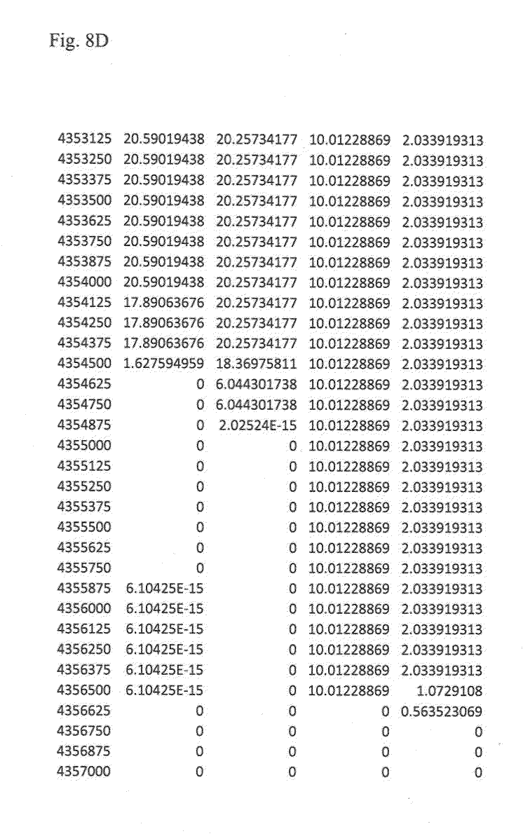

FIGS. 8A-8D. Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrated a region of the genome that contains the genes yjdL, cadA, and fumB from the 1.75 g/L SCALEs selection and analysis.

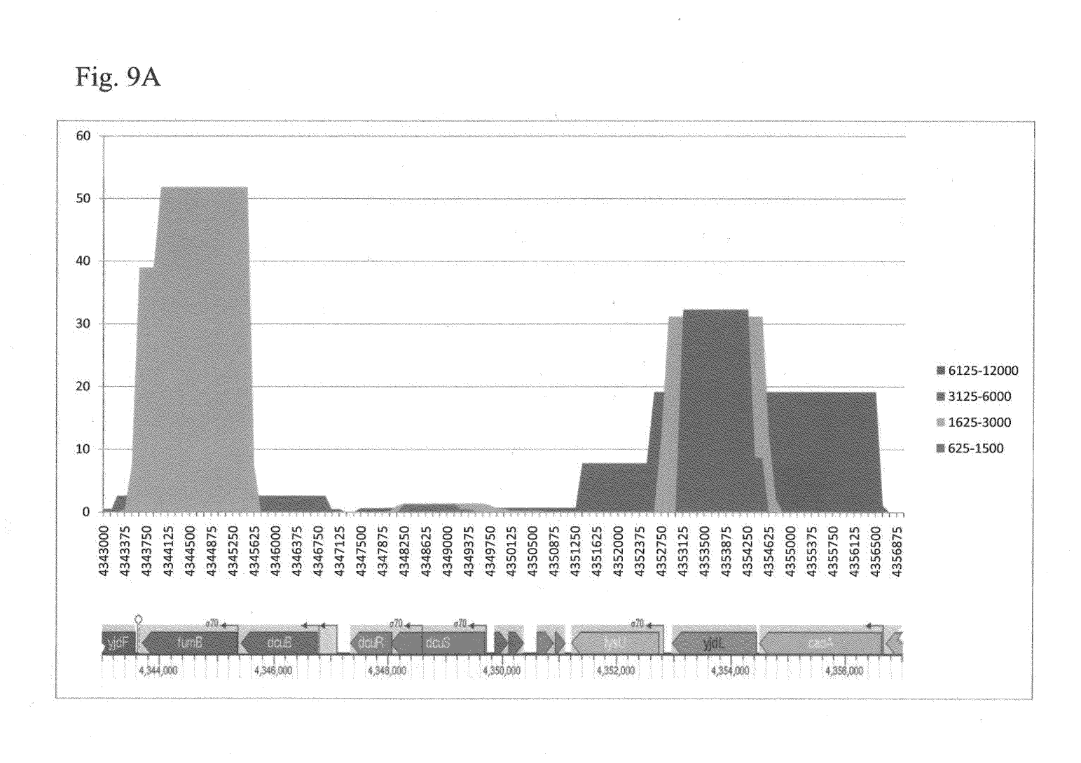

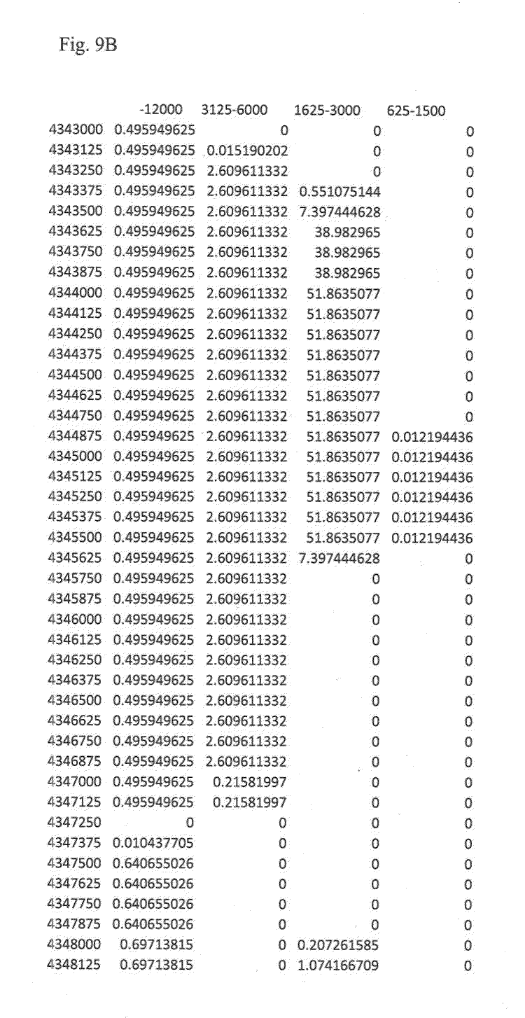

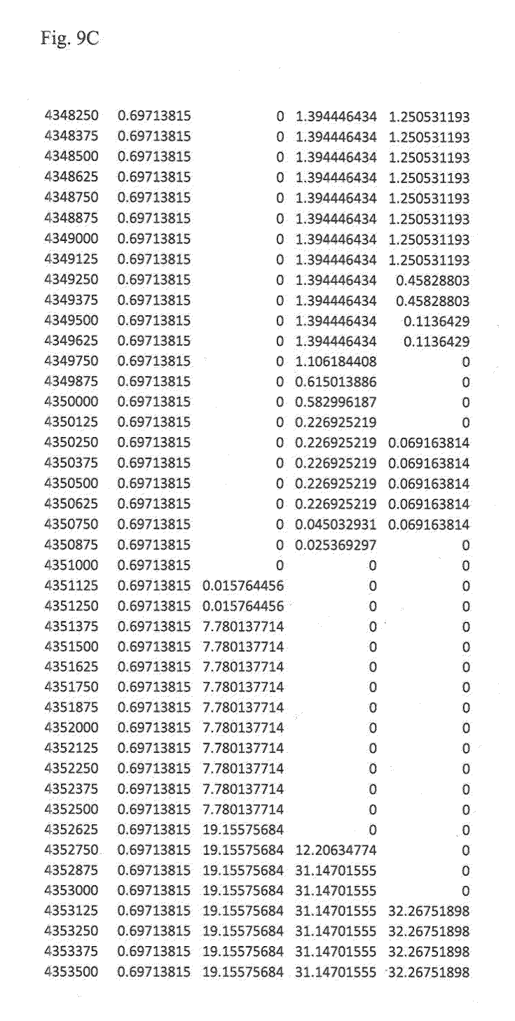

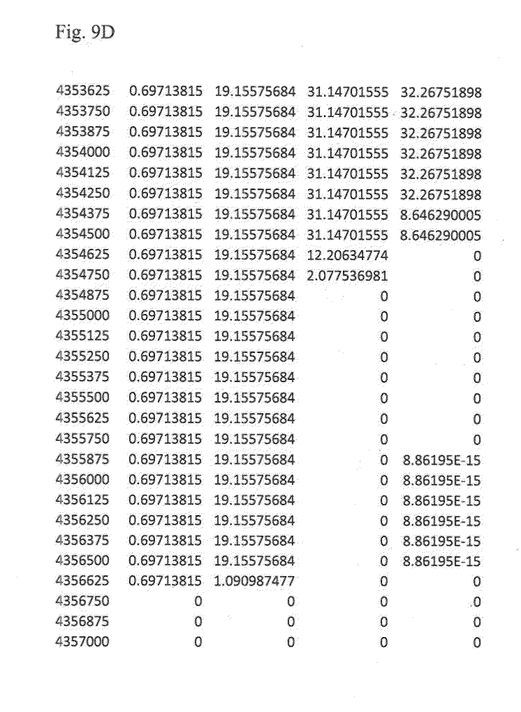

FIGS. 9A-9D. Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrated a region of the genome that contains the genes yjdL, cadA, and fumB from the 2.5 g/L SCALEs selection and analysis.

### Example 6

A pilot SCALEs selection was run on furfural tolerance. A SCALEs library was constructed in the pBTL-1 expression vector system. Cells were transformed with library plasmids and plated onto MOPS minimal media plates, supplemented with varying levels, 1-5 g/l, of furfural. Plates were incubated at 37° C. until colonies appeared, up to four days. Colonies were randomly selected to test for confirmation of improved tolerance phenotype.

Then, a SCALEs selection was performed in a similar fashion as the pilot selection, with the addition of microarray analysis, as prescribed by the SCALEs method. The expression vector system used for the SCALEs furfural selection was pSMART-LCK (Lucigen). Fitness values were calculated based on microarray analysis.

Certain experiments described herein were performed in E. coli BW25113 recA-, with the kanamycin resistance gene removed.

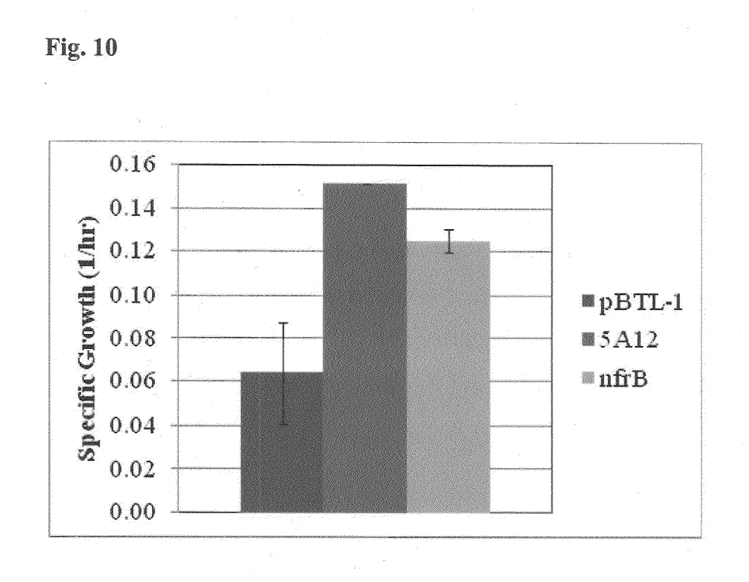

In one example, identification of gene nfrB as dominant contributing component towards tolerance from an exemplary clone, Clone 5A12. Cultures were grown in 5 ml MOPS minimal media, 100 μg carbenicillin/ml, and 1 g furfural/1 supplemented in 15 ml conical tubes at 37° C. The optical density of 1 ml culture was measured with a spectrophotometer at 600 nm. A natural log transformation of the data was then calculated and then a regression line was fit to the data to calculate specific growth, which is proportional to the culture doubling time. The blank vector, pBTL-1, served as the control. Clone 5A12 contains the gene nfrB as well as approximately 2200 bp of surrounding genomic sequencing. The nfrB subclone was produced by standard cloning techniques with primers designed to encode for the gene. The resulting cloned DNA was then ligated to pBTL-1 vector. FIG. 10 represents a histogram plot of data obtained from this clone. This data supports that the expression of nfrB contributes to aldehyde tolerance. Compared to control, Clone 5A12 has a higher specific growth. The nfrB subclone also contributes to increasing aldehyde tolerance.

### Example 7

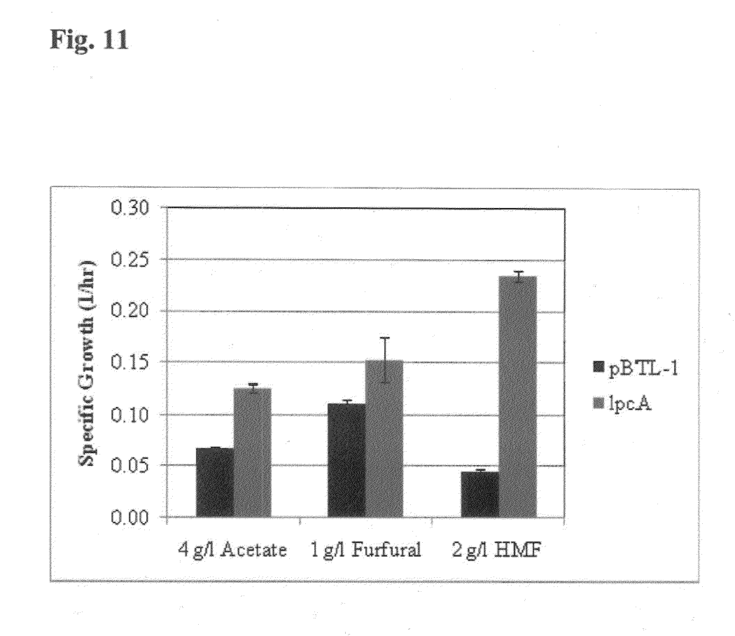

In other methods, a summary of the data in the tables obtained from studies performed herein is represented in FIG. 11. Here, confirmation of lpcA conferring tolerance to furfural and HMF (aldehydes) and acetate (acid) were observed. Cultures were grown in 10 ml MOPS minimal media, 100 μg carbenicillin/ml, and either 1 g furfural/1 supplemented, 2 g HMF/1, or 4 g acetate/1, in 15 ml conical tubes at 37° C. The optical density of 1 ml culture was measured with a spectrophotometer at 600 nm. A natural log transformation of the data was then calculated and then a regression line was fit to the data to calculate specific growth, which is proportional to the culture doubling time. The blank vector, pBTL-1, served as the control. The lpcA gene was cloned into the pBTL-1 using standard cloning techniques and primers designed to amplify the gene from template DNA.

This data supports that lpcA confers tolerance to a variety of low molecular weight organic compounds, both aldehydes and acids. Under all conditions tested, specific growth is improved when lpcA is overexpressed using the pBTL-1 vector system.

### Example 8

In another exemplary method, (data not shown) a circle plot after 72 hours in 1.75 g/L selection was compiled. Peaks represent clones found after SCALEs selection and analysis. Peak size represents relative fitness. Peak location represents location on E. coli genome. Peak color represents size of clone: blue 8 kb, green 4 kb, yellow 2 kb, and red 1 kb. In addition, growth rate of high-fitness clones compared to control were analyzed using various gene inductions (data not shown).

### Example 9

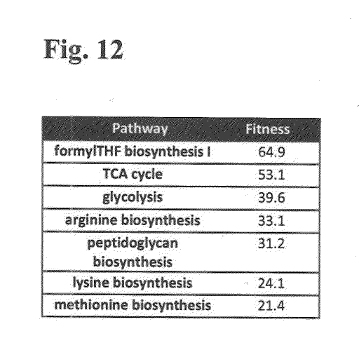

Top pathway fitness. Individual gene fitness were determined by analyzing the clones found in the SCALEs data (see FIG. 12). Multiple clones may contain the same gene or part of a gene. To calculate the gene fitness per clone, the clone fitness was multiplied by the fraction of the gene contained in the clone; this was then divided by the length of the gene. Once this was done for all clones that contained the gene, these were summed to yield the total gene fitness. This process was repeated for every gene in the ecocyc.org database for E. coli K12 MG1655. Subsequently, pathway fitness was determined by summing the individual gene fitness for all the genes in a particular pathway. Here, from the 1.75 g/L selection, formylTHF biosynthesis genes were found to be important to acetate tolerance. Arginine and methionine are also important pathway targets based on these findings.

### Example 10

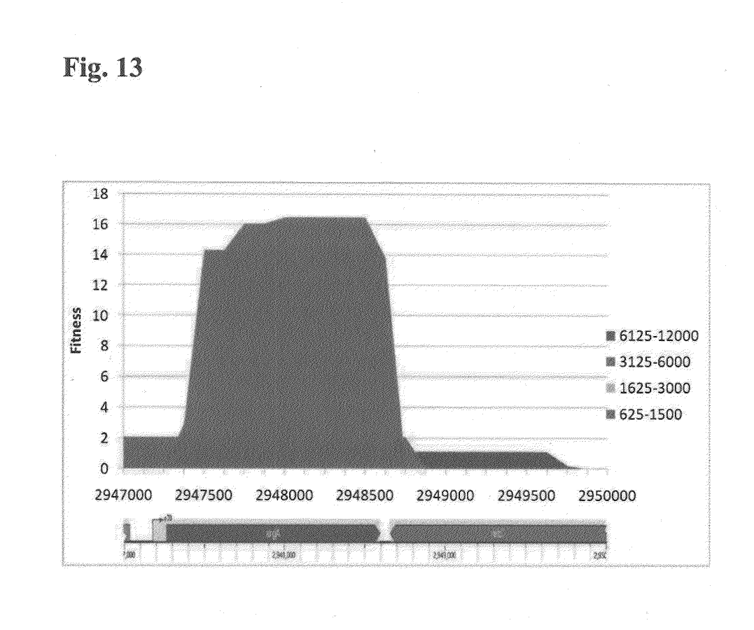

Blow-up regions of the E. coli genome were evaluated and plotted (see FIG. 13). Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrate, the region of the genome that contains the gene argA from the 1.75 g/L SCALEs selection and analysis.

### Example 11

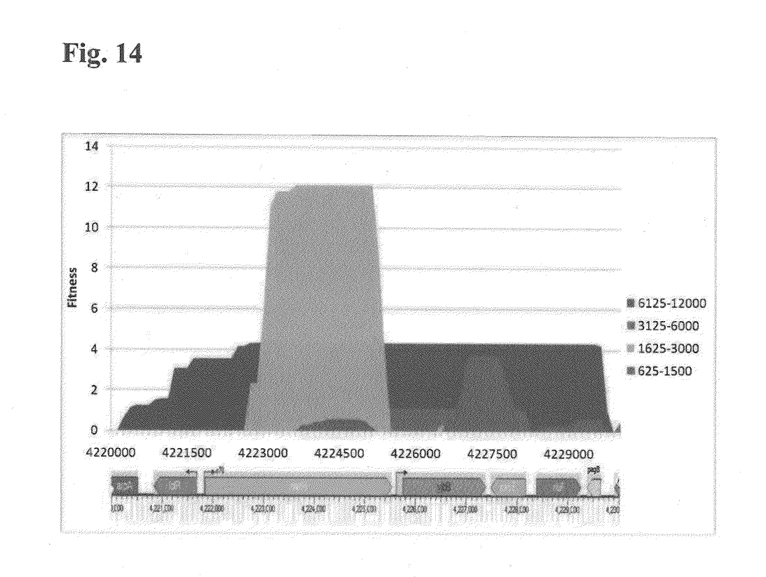

Blow-up regions of the E. coli genome were evaluated and plotted (see FIG. 14). Clone fitness mapped over E. coli genome. Peak size is relative to fitness. Colors denote the size of the clones: Blue 1 kb, Red 2 kb, Green 4 kb, and Purple 8 kb. In detail, fitness value plotted over genomic position. Here is illustrate, the region of the genome that contains the gene metH from the 1.75 g/L SCALEs selection and analysis.

### Example 12

FIG. 15 represents results from pilot selection. Randomly selected clones from the furfural aldehyde selection demonstrate a high frequency for the lgt-thyA operon.

TABLE 1
1.75 g/L Acetate Selection

| | | | | | |
|---|---|---|---|---|---|
| 100625 | 101875 | 1250 | 22.84 | murC^{†}, murG | UDP-N-acetylmuramate-alanine ligase |
| 4353125 | 4354625 | 1500 | 15.19 | yjdL^{†}, cadA | dipeptide transporter |
| 4352875 | 4354625 | 1750 | 12.33 | yjdL*, cadA | dipeptide transporter |
| 2947500 | 2948750 | 1250 | 11.39 | argA^{†}, recD | N-acetylglutamate synthase |
| 4344000 | 4345750 | 1750 | 8.29 | fumB^{†}, dcuB | fumarase B |

2.5 g/L Acetate Selection

| | | | | | |
|---|---|---|---|---|---|
| 4353125 | 4354625 | 1500 | 32.27 | yjdL^{†}, cadA | dipeptide transporter |
| 4343625 | 4345625 | 2000 | 31.59 | fumB^{†}, dcuB | fumarase B |
| 4352875 | 4354625 | 1750 | 18.94 | yjdL*, cadA | dipeptide transporter |
| 4344000 | 4345750 | 1750 | 12.88 | fumB^{†}, dcuB | fumarase B |
| 4352625 | 4356625 | 4000 | 11.38 | yjdL*, cadA^{†} | dipeptide transporter, |
| 100625 | 101875 | 1250 | 11.13 | murC^{†} | UDP-N-acetylmuramate-alanine ligase |

*the entire gene is present in the clone.
^{†}means that >80% of the gene is present in the clone

TABLE 2

| | | | |
|---|---|---|---|
| Gene Name | Fitness | Gene Product | Pathway |

1.75 g/L Selection

| | | | |
|---|---|---|---|
| Yjdl | 50.3 | dipeptide transporter | |
| murC | 23.7 | UDP-N-acetylmuramate-alanine ligase | peptidoglycan biosynthesis III |
| fumB | 19.6 | fumerase B | TCA cycle |
| cadA | 15.1 | lysine decarboxylase | lysine degradation |
| argA | 14.9 | N-acetylglutamate synthase | arginine biosynthesis |
| meth | 13.7 | homocysteine transmethylase | formylTHF biosynthesis 1 |

2.5 g/L Selection

| | | | |
|---|---|---|---|
| YYjdl | 78.0 | dipeptide transporter | |
| murC | 11.9 | UDP-N-acetylmuramate-alanine ligase | peptidoglycan biosynthesis III |
| fumB | 21.2 | fumerase B | TCA cycle |
| cadA | 24.7 | lysine decarboxylase | lysine degradation |
| lpcA | 9.7 | D-sedoheptulose 7-phosphate isomerase | ADP-L-glycero-b-D-manno-heptose |
| | | | biosynthesis |

TABLE 3
Acid Tolerance (Acetate Selection): Data
from aerobic growth in 4 g/L acetate

| | | Specific | | | Final | |
|---|---|---|---|---|---|---|
| | SCALEs | Growth | Standard | | OD₆₀₀ | |
| Gene | Fitness | (hr⁻¹) | Deviation | n | (24 hr) | Comment |
| pBTL-1 | N/A | 0.067 | 0.001 | 3 | 0.111 | |
| Control | | | | | | |
| pBTL-1 | Get from | 0.126 | 0.004 | 3 | 0.324 | SCALEs |
| lpcA | Nich | | | | | selection |
| | | | | | | performed in |
| | | | | | | pBTL-1 vector |

TABLE 4
Aldehyde Tolerance (Furfural Selection): Data
from aerobic growth in 1 g/L furfural

| | | Specific | | | Final | |
|---|---|---|---|---|---|---|
| | SCALEs | Growth | Standard | | OD₆₀₀ | |
| Gene | Fitness | (hr⁻¹) | Deviation | n | (24 hr) | Comment |
| pBTL-1 | N/A | 0.111 | 0.003 | 3 | 0.586 | |
| Control | | | | | | |
| pBTL-1 | 11.9 | 0.153 | 0.022 | 3 | 0.918 | SCALEs |
| lpcA | | | | | | selection |
| | | | | | | performed in |
| | | | | | | pSMART LCK |
| | | | | | | vector, confir- |
| | | | | | | mation studies |
| | | | | | | performed in |
| | | | | | | pBTL-1 |

TABLE 5
Aldehyde Tolerance (Furfural Selection): Data from
aerobic growth in 2 g/L hydroxymethyl furfural

| | | Specific | | | Final | |
|---|---|---|---|---|---|---|
| | SCALEs | Growth | Standard | | OD₆₀₀ | |
| Gene | Fitness | (hr⁻¹) | Deviation | n | (24 hr) | Comment |
| pBTL-1 | N/A | 0.045 | 0.002 | 3 | 0.170 | |
| Control | | | | | | |
| pBTL-1 | See fit- | 0.234 | 0.005 | 3 | 1.014 | SCALEs |
| lpcA | ness for | | | | | selection |
| | furfural | | | | | performed in |
| | selection | | | | | pSMART LCK |
| | | | | | | vector, confir- |
| | | | | | | mation studies |
| | | | | | | performed in |
| | | | | | | pBTL-1 |

### Example 13

In one exemplary method, it was demonstrated that efforts resulting in the confirmation that increased dosage of lpcA confers tolerance to all three compounds, the elucidation of the mechanistic basis for the pLPCA tolerant phenotypes in the selection host strain, and the identification of AraC as a potential regulator of LpcA.

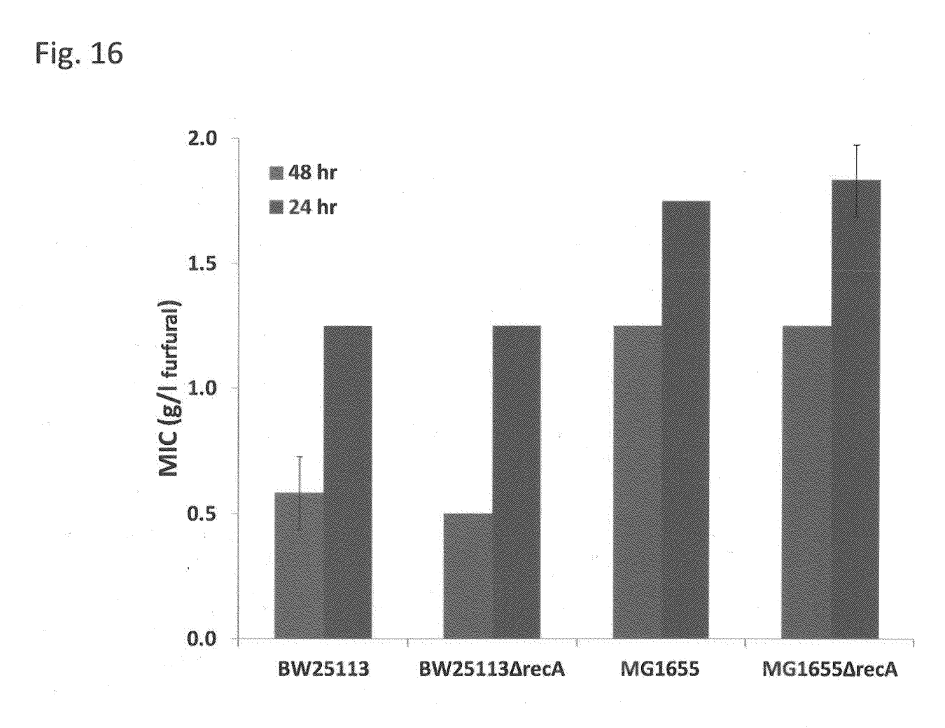

In one exemplary method, assays were performed to determine minimum inhibitory concentration (MIC) of ethanol, furfural and acetate for BW25113 and MG1655 E. coli strains. FIG. 16 represents a histogram plot of illustrating tolerance differences of furfural in BW25113 and MG1655 E. coli strains. Cells from each strain were inoculated from the same optical density into a total of 100 μL of varying furfural concentrations in a minimal media, for example using MOPS. Cell growth in the first 24 hr or 48 hr in the presence of furfural was monitored. The left bar in each pair for each indicated strain represents a first concentration of furfural that inhibited bacterial growth after 24 hr. The right bar for each strain represents the first concentration that inhibited growth after 48 hr. The MIC reads were the average of triplicates (n=3), and error bars represent standard deviation. It was demonstrated that furfural MIC of MG1655 E. coli strain was significantly higher than BW25113, 1.2 g/L vs. 0.5 g/L after 24 hr and 1.8 g/L vs. 1.2 g/L after 48 hr. Thus, this difference in strains led to further investigation into the difference between these strains. ArecA signifies strains where the gene has been knocked out as previously described.

### Example 14

### Genome-Wide Identification of lpcA as a Commonly Enriched Locus for Tolerance

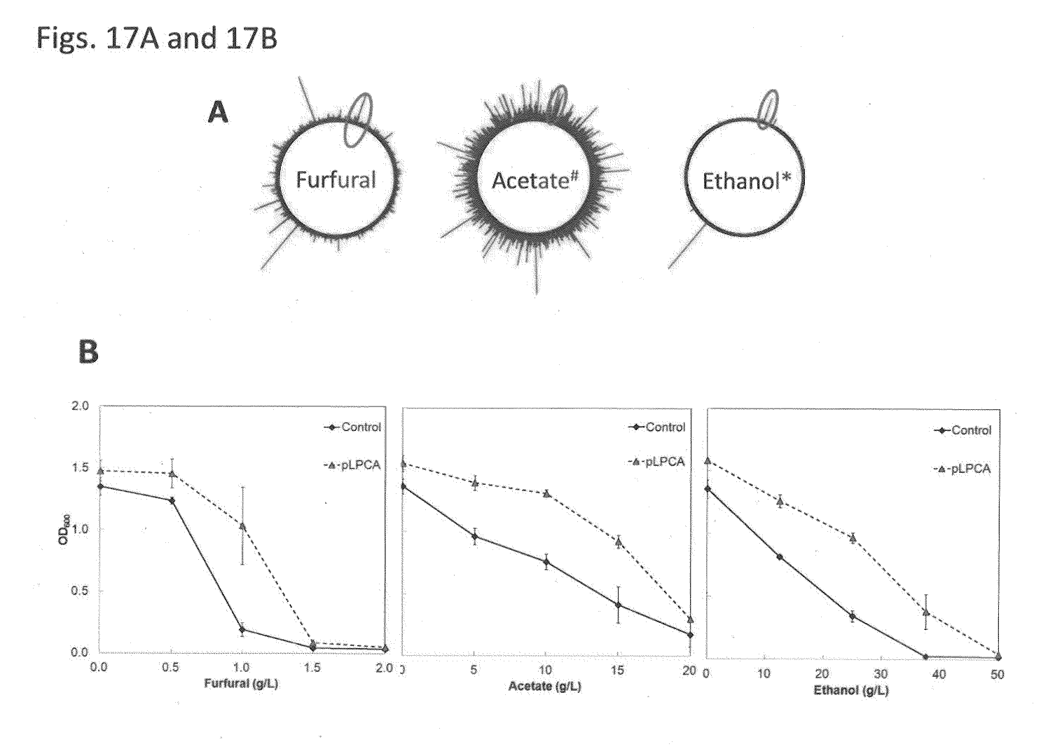

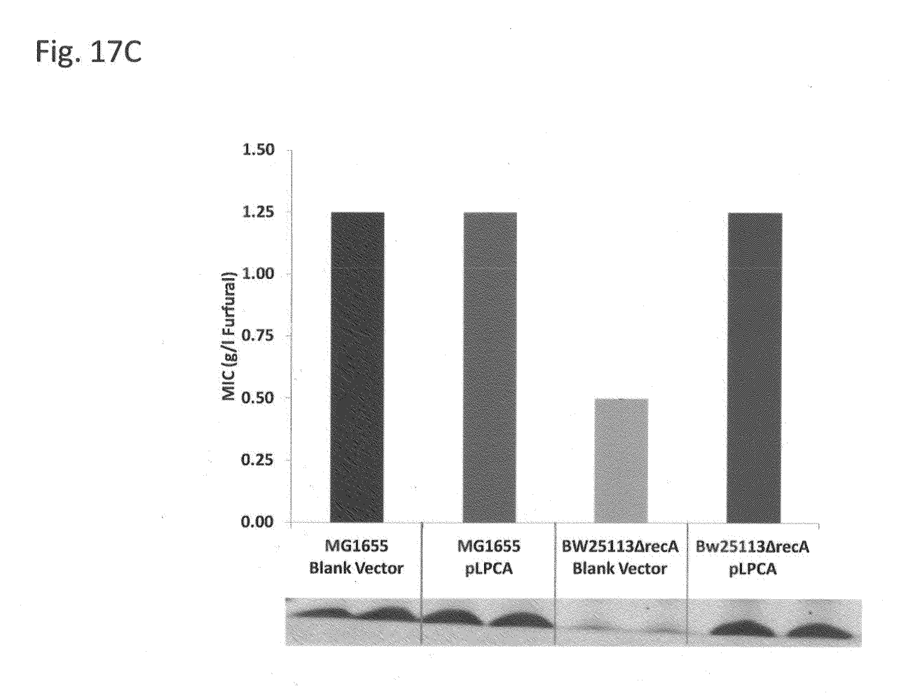

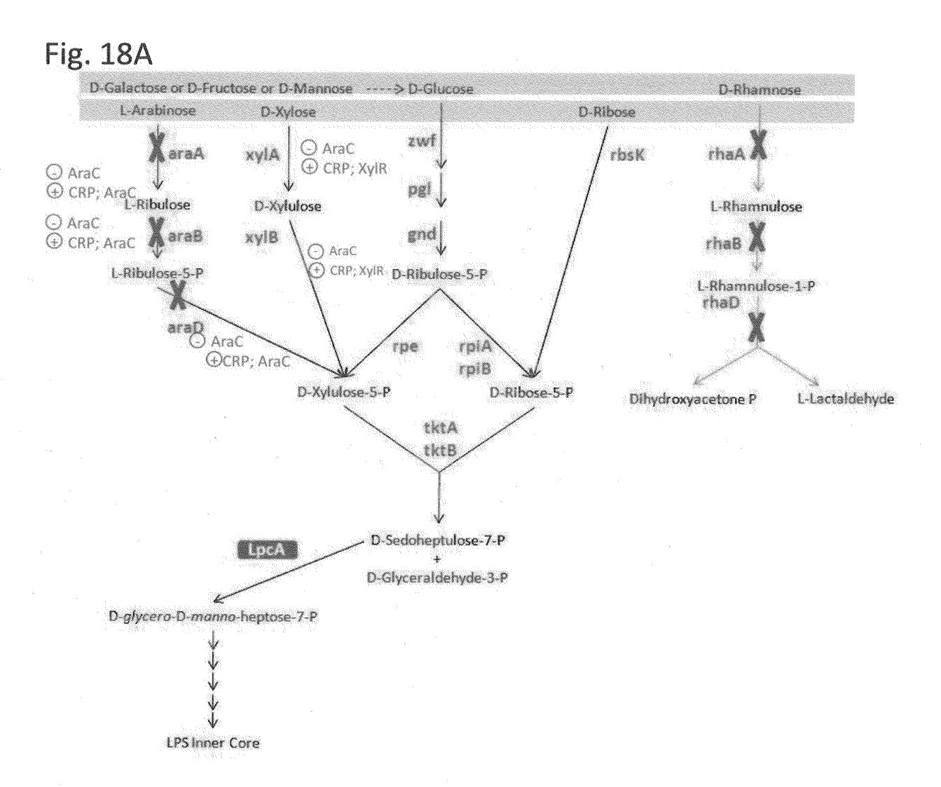

The SCALEs method combines plasmid-based genomic libraries of multiple insert sizes, DNA micro-array based quantification of insert DNA, and a mathematical signal decomposition approach to track several hundred thousand library clones in parallel throughout growth selections or other high throughput screens. Here, the SCALEs method was employed to track enrichments of E. coli genomic libraries throughout a growth selection in the presence of inhibitory levels of furfural. This selection resulted in the identification of a broad range of genes (FIG. 17A) whose overexpression resulted in improved growth, and thus implicates these genes in furfural tolerance (further study of these genes is the focus of another effort). Libraries were grown on solid minimal media supplemented with furfural to spatially isolate individual clonal tolerance due to the ability of E. coli to reduce furfural to the less-toxic furfuryl alcohol through various oxidoreductases (previously disclosed). When comparing the furfural data to those obtained from SCALEs selections performed for acetate (previously presented) and ethanol tolerance, it was observed that increased dosage of the lpcA locus was implicated in tolerance in all three studies (FIG. 17A). Not only was overlapping enrichment via the SCALEs-based microarray results, but also via sequencing of clones isolated from each of the selections' end population.

FIGS. 17A-17B represent methods of identification of a gene related to restoring tolerance. Multiple genome-wide SCALE selection was performed in the presence of ethanol, furfural and acetate respectively. The data set obtained for each compound was compared to the others. Alleles enriched in all cases were identified. FIG. 17A demonstrated circle plots showing end-point frequency of alleles across the E. coli chromosome after selection using the SCALEs method, among which lpcA allele was marked as an example. To restore tolerance, overexpression of identified genes in bacterial cells was performed. For example, lpcA ORF was cloned into the pBTL-1 vector. BW25113 ArecA cells transformed with the blank pBTL-1 vector were used as a control. BW25113 ArecA cells overexpressing lpcA gene were referred to as pLPCA. Cells were inoculated at a standard cell density into MOPS minimal media, supplemented with furfural, acetate or ethanol. After 24 hours, growth of the cells was analyzed. As demonstrated in FIG. 17B, with overexpression of lpcA gene, growth of bacterial cells in the presence of 0-2 g/L of furfural, 0-20 g/L of acetate or 0-50 g/L of ethanol was significantly increased. For example, in the presence of 1 g/L of furfural controls cells at 24 hr showed OD600 of about 0.2, while pLPCA cells showed about 1.2; in the presence of 10 g/L of acetate, control 0.75, pLPCA 1.4; and in the presence of 25 g/L of ethanol, control 0.4 and pLPCA 1.0.

The minimum inhibitory concentration (MIC) of furfural of MG1655 and BW25113 with or without lpcA overexpression was determined. MIC assays were performed as described in Example 13. As demonstrated in FIG. 17C, MG1655 showed higher tolerance to furfural (MIC of 1.25 g/L) compared to BW25113 ArecA (MIC of 0.5 g/L). With overexpression of lpcA, tolerance of BW25113 ArecA pLPCA (MIC of 1.25 g/L) increased to a comparable level of tolerance of MG1655. MIC reads were average of triplicates (n=3), and error bars represent standard deviation. The SDS-PAGE gel image in FIG. 17C represents LPS level of the cells indicated above the gel image (duplicate of each cell sample was presented). LPS was extracted from the indicated cells grown to exponential phase. Loading of LPS the SDS-PAGE gel was normalized to the cell number. The gel was stained for visualization after desired separation of LPS was obtained. BW25113 ArecA transformed with the blank vector demonstrated lowest LPS expression level. Overexpression of lpcA gene efficiently restored the expression of LPS in the BW25113 ArecA cells to the comparable level demonstrated by MG1655 cells (with or without overexpression of lpcA gene).

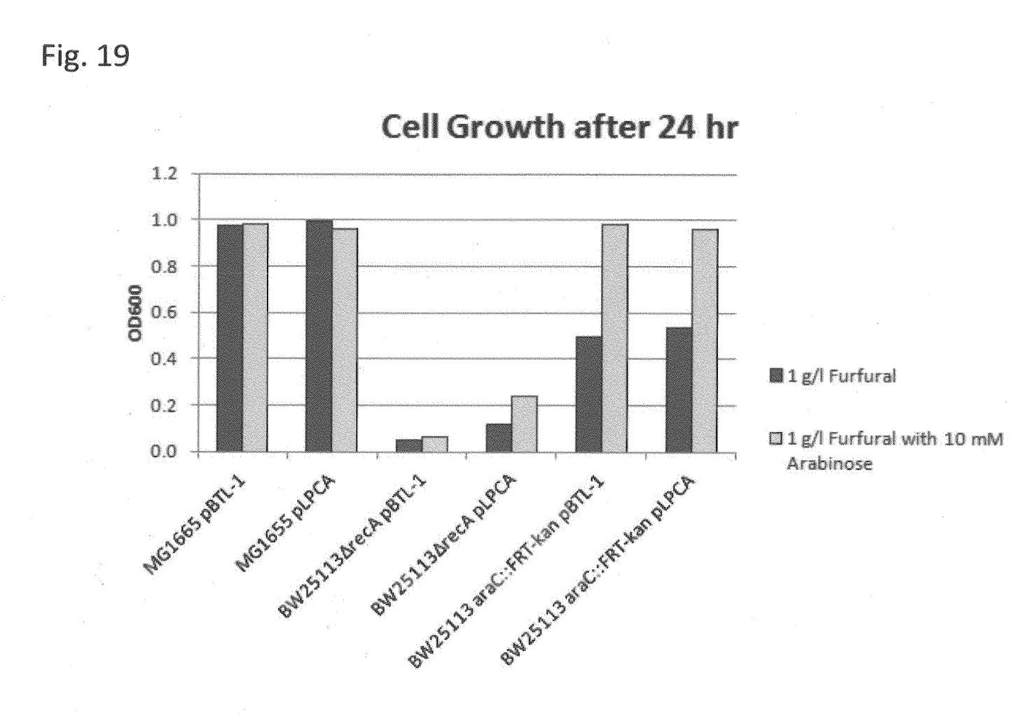

Based on these observations, lpcA ORF was cloned into the pBTL-1 vector, creating pLPCA, and performed tolerance assessments in furfural (1 g/l), acetate (5 g/l), and ethanol (25 g/l) (FIG. 19). In all three inhibitory conditions, pLPCA was confirmed to improve the final optical density in BW25113: 252±8% in furfural, 47±4% in acetate, and 37±3% in ethanol.

### BW25113 is Less Tolerant Relative to MG1655

As a further step in validation, pLPCA was transformed into E. coli MG1655 and measured growth in the presence of furfural, acetate, and ethanol. Surprisingly, no increase in tolerance to any of the three compounds was observed in MG1655 with pLPCA (p-value >0.1 for all conditions) (FIG. 19A). Rather, MG1655 appeared to display increased tolerance relative to BW25113 such that the pLPCA conferred tolerance phenotype was lost. Wild-type MG1655 was significantly more tolerant than wild-type BW2511 in each case (FIG. 19B).

LpcA catalyzes the first committed step towards the heptose component of inner core LPS, a vital component of the outer membrane. While the compounds studied each have varying effects on outer membrane stability, it is known that increased hydrophobicity is positively correlated with toxicity for all three of these chemical families (previously presented). These results indicate that lpcA overexpression increases cellular resistance to furfural, the most hydrophobic of the compounds tested here (logP_{octanol/water} of 0.41).

pLPCA Benefit

It was demonstrated that there is difference in LPS formation associated with altered tolerance phenotypes between MG1655 and BW25113 with and without LpcA overexpression. E. coli MG1655 and BW25113 are both K-12 derivatives and their genotypes vary by only four mutations: A(araBAD)567, A(rhaBAD)568, AlacZ4784(::rrnB-3), hsdR514 (Baba et al. 2006). However, to provide more support for this hypothesis, and assess any predicted flux through the PPP, modeled predicted fluxes of BW25113 and MG1665 on various carbon sources (glucose, ribose, and xylose) feeding into the PPP, which supplies S7P. Interestingly, fluxes from S7P towards LPS synthesis were identical for BW25113 and MG1665 on glucose, with both predicting 0.023 mmol/gDW/h (see Supplementary Material, FIG. 1, for BW25113 fluxes).

Because varied stoichiometric modeling did not explain the difference in LPS formation observed in vivo, regulatory effects were considered by assessing the impacts of the individual deletions in BW25113 compared to MG1665. Due to the known regulatory effects of AraC on the araBAD and xylAB operons, which both act on pentose sugars that feed directly into the PPP and S7P formation, it was hypothesized that AraC was playing a role in modulating LPS synthesis that had not previously been reported. AraC regulates the expression of the araBAD operon, which is deleted in BW25113, therefore its function as a regulator might differ in an araBAD mutant (similar to the bimodal response of AraC control seen in a P_{araBADT}-plasmid system at subsaturating inducer levels. It was thought that AraC may have a regulatory affect on LPS formation (at least one that is observable in an araBAD mutant like BW25113). It was hypothesized that AraC has a regulatory function towards LPS formation in BW15113, and that either lpcA overexpression via pLPCA or deletion of araC can overcome this negative regulatory effect. This hypothesis was examined by measuring a BW25113 araC deletion mutant, JW1063, for inhibitor tolerance and LPS formation. As shown in FIG. 19, JW0063 in fact displays increased tolerance to each of the three compounds relative to BW25113. In all cases, pLPCA expressed in JW0063 did not yield growth improvements compared to vector control. These data indicate that restoration of tolerance in BW25113 can be obtained through one of two routes: overexpression of lpcA or deletion of araC, where araC deletion appears to be the more effective approach.

### Example 15

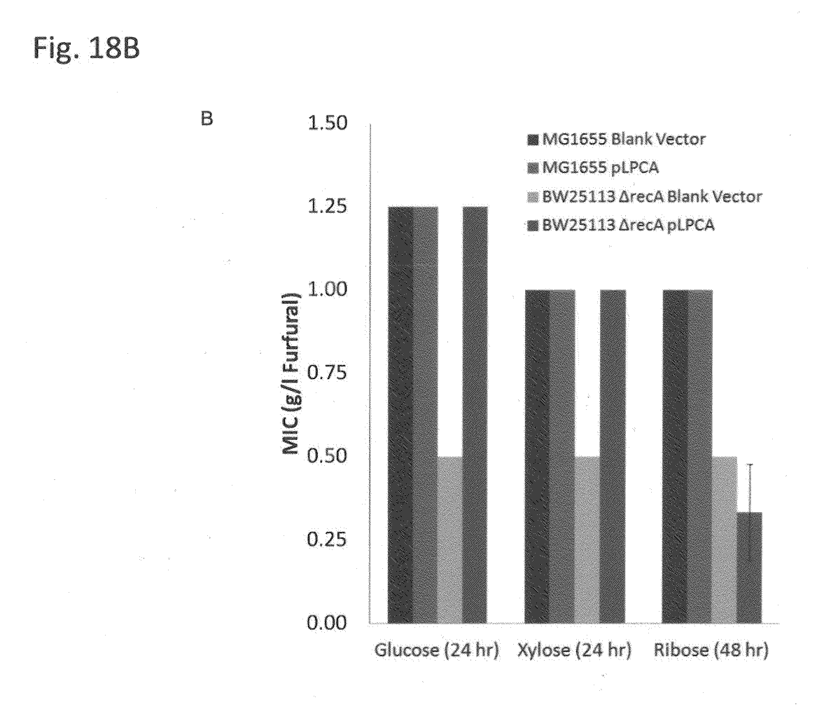

A schematic of LPS inner core biosynthesis and genetic differences between MG1655 and BW25113 is provided by FIGS. 18A-18B. E. coli metabolism pathways leading to formation of sedoheptulose-7-phosphate (S7P) from various sugar sources are demonstrated. S7P serves as the metabolite which is converted by LpcA in the first committed step towards LPS inner core biosynthesis. Known points of regulation by AraC are also indicated (regulatory annotations were taken from ecocyc.org).

In another exemplary method, MIC was determined when MG1655 and BW25113 strains were fed with various carbon sources. MIC assays were performed in the presence of furfural. Glucose, Xylose or Ribose was supplemented with a minimal media for cell growth. FIG. 18B illustrates an example of the effect of pLPCA overexpression on cell growth in various conditions. MICs were read at the indicated time (24 hr or 48 hr), the average of triplicates was presented and error bars represent standard deviation. ‘Blank vector’ refers to the pBTL-1 vector, which served as the control. Overexpression of lpcA gene increased tolerance of BW25113 ArecA significantly with either Glucose or Xylose as a carbon source. BW25113 ArecA transformed with the blank vector showed MIC of 0.5 g/L for both glucose and xylose, while BW25113 ArecA pLPCA 1.25 g/L for glucose and 1.0 g/L for xylose, which was comparable to MG1655 with or without overexpression of lpcA gene. The phosphorylated form of the ribose, ribose-5-phosphate, is a known inhibitor in the reaction pathway towards 3-deoxy-D-manno-octulosonate 8-phsophate phosphatase, KdsA, which is a metabolite in the pathway towards LPS synthesis.

### Example 16

In other exemplary methods, tolerance and expression of LPS was restored in a microorganism. FIG. 19 represents an example of restoration of tolerance and LPS expression in BW25113 ArecA clone. The indicated cells were grown in MOPS minimal media supplemented with glucose as a carbon source. Furfural was added to the media at the concentration of 1 g/L. Arabinose was added at 10 mM where indicated to gauge effect of AraC binding. The BW25113 araC::FRT-kan clone corresponds to JW0063, a BW25113 araC knockout mutant ordered from the Keio Collection directly. pBTL-1 represented the blank vector, and pLPCA referred to lpcA gene overexpression. Consistent with the Examples above, overexpression of lpcA gene increased cell growth (OD600 of 0.1 vs. 0.05) in the presence of furfural. Addition of Arabinose to the lpcA gene overexpression cells could further enhance the effect (OD600 of 0.3). A dramatic increase of cell growth against inhibition of furfural was demonstrated by the BW25113 araC::FRT-kan clone (OD600 of 0.5) without overexpression of lpcA gene. Addition of Arabinose to the culture of the BW25113 araC::FRT-kan clone could significantly augmented cell growth (OD600 of 1.0). A combination of deletion of araC and addition of Arabinose to the culture could restore tolerance to furfural of BW25113 ArecA to the comparable level demonstrated by MG1665 (MG1665 with or without overexpression of lpcA gene in the presence or absence of Arabinose). Combination of araC deletion and overexpression of lpcA gene also increased cell growth: OD600 of 0.5 in the absence of Arabinose; and OD 600 of 1.0 in the presence Arabinose. To analyze the LPS level in the cells indicated in FIG. 19, LPS are extracted from the cells grown to exponential phase. Loading of LPS for the protein gel is normalized to the cell number. The gel is stained to visualize LPS protein bands after desired separation is obtained. The LPS level in the araC knockout mutant clone is compared to other clones to demonstrate restoration of LPS by deletion of araC.

Overexpression of lpcA or Deletion of araC Restores LPS Formation in BW25113

In another exemplary embodiment, LPS formation in the wild-type strains and with pLPCA. Carbohydrate staining of extracted LPS (data not shown) confirmed that BW25113 is deficient relative to MG1655, and that both of the genetic modifications elucidated here for tolerance restoration also restore LPS formation (data not shown). Because the stain focuses on oxidized carbohydrate groups, the LPS profiles support that MG1665 and the other tolerant strains contain more sugar components in their LPS cores, which corresponds the role of LpcA in heptose formation. It is important to note that BW25113 has been shown to express detectable levels of LPS core structures, but this data indicates that BW25113 is deficient, relative to MG1665, to a degree such that chemical tolerance is significantly impacted.

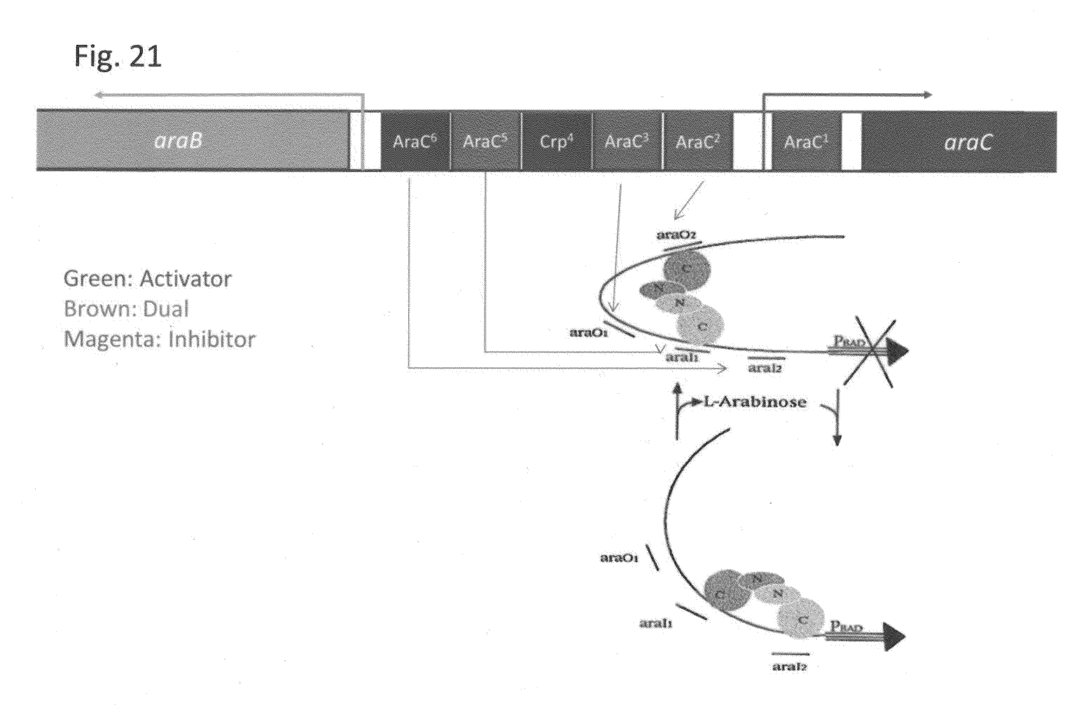

AraC is documented to control six transcriptional units through 13 different binding sites in E. coli MG1655: araBAD, araC, araJ, araFGH, araE, and xylAB. Multiple AraC binding sites are encoded around these transcripts; for example, five AraC binding sites exist between the araBAD and araC operons (AraC can bind as a dimer) to repress or induce transcription. In fact, different sequences have been reported as the consensus sequence for binding sites in this family as more sites are discovered in E. coli and compared to other organisms. It was considered that homology to known AraC binding sites are upstream of the lpcA ORF. To test this hypothesis, the sequences upstream of the lpcA promoter were compared to the five AraC binding sites that regulate araBAD and/or araC expression (FIG. 21). It was observed that binding sites AraC3 and AraC5 demonstrated 7 bp and 8 bp sequential homologies to the queried sequence, respectively. There is a <1% chance of randomly finding the 8 bp sequential homology alignment observed here. Moreover, the sequential homology aligned for the sequence upstream of the lpcA promoter is greater than that existing between some of the known AraC sites with each other (data not shown).

It is possible that other sequences with high-homology to AraC binding sites might be found outside of the searched region (as a second, more distal site would allow for the DNA looping repression mechanism observed for araBAD regulation). In addition, RhaS and RhaR, which are members of the AraC regulatory family are involved in the regulation of rhamnose metabolism via rhaBAD (RhaS as a direct regulator, and RhaR by regulating RhaS), which is another deleted operon in BW25113. So the possibility exists that a similar mechanism might be acting through the rhamnose regulators.

### Example 17

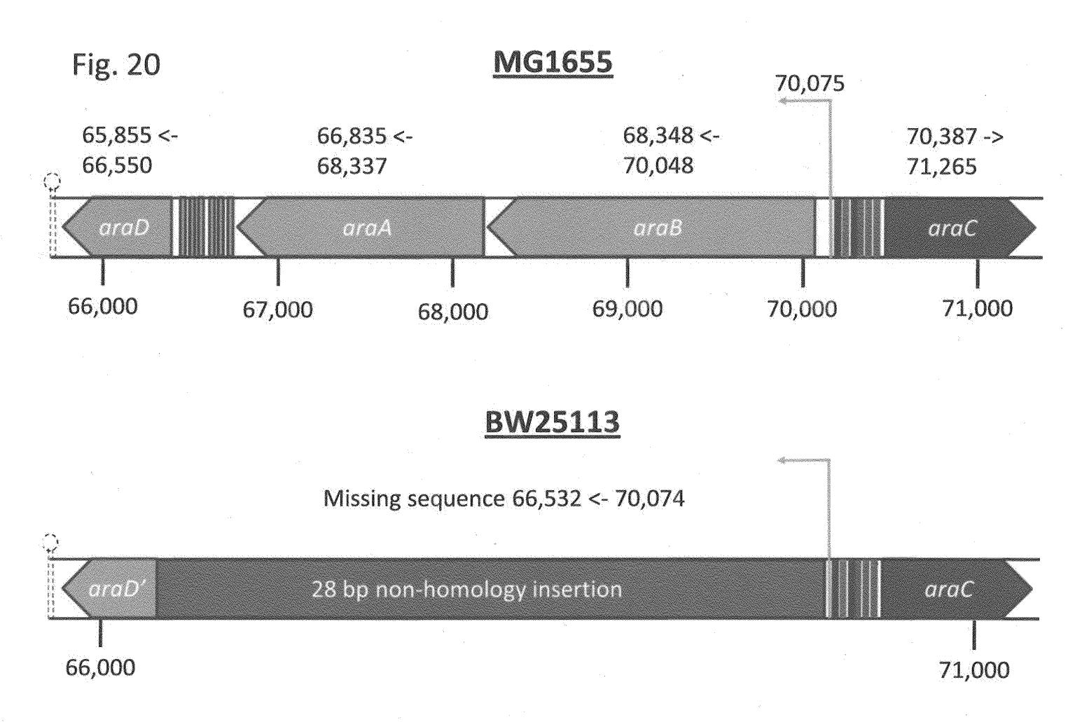

FIG. 20 represents sequencing results from in-house amplification of araBAD operon and araC loci. Known gene lengths are indicated (numbers correspond to the by annotation according to ecocyc.org). Differences of the araBAD operon between MG1655 and BW25113 are demonstrated.

### Example 18

FIG. 21 provides a schematic to demonstrate a blowup region of known binding sites of AraC regulating the araBAD operon. The araBAD deletion in BW25113 did not remove the sequence of these known binding sites. The araC knockout mutant, JW0063, is missing site AraC', the binding site downstream of the promoter. The araC induction model provided by Gallegos et al., (MMBR, 1997) was incorporated for demonstration purpose.

### Materials and Methods

### Bacterial Strains and Media

E. coli

BW25113, MG1655, and derivatives were used where indicated. JW0063, an araC mutant of BW25113 was obtained from the Coli Genetic Stock Center (Yale). Overnight bacterial cultures were grown in Luria-Bertani medium with shaking. MOPS minimal medium (previously presented) with 0.2% glucose as a carbon source, was used for all seed cultures and growth studies. Seed cultures were inoculated at 2 v/v % from an overnight culture and were grown to mid-exponential phase. Growth cultures were inoculated with 10 v/v % seed cultures of an OD₆₀₀ of 0.195-0.200 for an initial optical density of ˜0.02. Carbenicillin and kanamycin were used where appropriate at 100 μg/ml and 30 μg/ml, respectively. The pBTL-1 vector (previously described) was used as the control blank vector for all growth studies. Genome-wide furfural selection

Genomic libraries of 1, 2, 4, and 8 kb cloned it the pSMART LC-Kan vector (e.g. Lucigen) were prepared as previously described (previously described). Library plasmids were transformed into E. coli BW25113 ArecA::FRT, which was prepared accordingly (previously described). After 1 hr recovery 1 ml in Terrific Broth media, dilutions of 1/1000 volume aliquots of each library transformation were plated on LB agar with kanamycin in triplicate to ensure transformation efficiency, as required for complete library coverage (previously described). The libraries were combined into a single culture and aliquoted onto MOPS minimal agar as a control or MOPS minimal agar plates supplemented with 0.75 g/l furfural. Plates were incubated until growth appeared on the control and furfural plates, one day and three days, respectively. Plates were scraped and plasmids were extracted using a QIAprep Spin Midiprep Kit (Qiagen). Microarray and data analysis were performed in a consistent manner as the acetate selection (previously described).

### Cloning

The lpcA gene was amplified via PCR from K-12 genomic DNA using the primers 5′-AAAGCTCACATTGTTGCTGTTTTTATC-3′ (SEQ ID NO: 6) and 5′-GAAGATTGATTTAAGAATTTT CAGGTCG-3′ (SEQ ID NO: 7). The purified product, which contained the entire ORF, was ligated to the blunt-ended pBTL-1 vector (Lynch and Gill 2006), creating pLPCA. The pBTL-1 vector was the vector used for library construction in the acetate SCALEs selection (previously described).

### LPS Extraction and Visualization

LPS was extracted from cultures grown into mid-exponential phase in MOPS minimal media. Cells were harvested and normalized against the lowest optical density for all samples (OD₆₀₀˜0.6). LPS was extracted using an LPS Extraction Kit (Intron Biotechnology). Samples were run on a Mini-ProTEAN® TGX Gel (Bio-Rad). The gel was stained using the Pro-Q® Emerald 300 Lipopolysaccharide Gel Stain Kit (Invitrogen), which stains for oxidized carbohydrate groups.

### Constraint-Based Modeling

The iJR904 genome-scale metabolic model of E. coli (Reed et al. 2003) was used to predict fluxes for MG1665 and BW25113 on different substrates. A modified model based on iJR904 was created by removing reactions corresponding to the functions of araBAD, rhaBAD, and lacZ (hsdR is not represented in the model). Maximum specific glucose uptake rate was set at 10 mmol/gDW/h and the specific uptake rate of pentose sugars was set to an equivalent carbon uptake of 12 mmol/gDW/h to minimize any effect on fluxes due to the lower carbon uptake. Simulations to maximize biomass were run using the COBRA toolbox (Becker et al. 2007) in Matlab (MathWorks, Natick, Mass.).

### Sequence Alignment for AraC Binding Sites

The 63 bp sequence between the upstream gene's proposed promoter (fadE is oriented counter-clockwise) and the proposed lpcA promoter was aligned with the five annotated AraC binding sequences (sequences were retrieved from ecocyc.org) using the alignment tool in A Plasmid Editor (Wayne Davis, University of Utah).

### Furfural75 Selection Description

A furfural selection was performed using the TRMR method. ‘Up’ and ‘Down’ libraries were mixed with the control strain at appropriate conditions and then aliquoted onto MOPS minimal media agar supplemented with 0.75 g/L furfural. Plates were grown at 37° C. until colonies appeared. Colonies were harvested from the plates and isolated by centrifugation. Genomic DNA was extracted and processed for microarray analysis according to the TRMR method.

### Data Set Preparation

Fitness values were calculated by dividing the frequency of the a given allele to that of the frequency of the control chip. In some cases certain alleles exceeded the maximum value on the calibration curve. The natural-log values were then calculated for these fitnesses. An allele was scored as enriched if ln(Fitness)>0. Roughly 100-200 genes (individually for ‘Up’ and ‘Down’ alleles) were found to be enriched for the acetate selection. Over 600 genes were enriched within the furfural selection, but values were included only for those with ln(Fitness)>2.

TABLE 6

| Genetic Variation | Gene Product |
|---|---|
| Δ(araBAD)567 | L-Arabinose degradation |
| Δ(rhaBAD)568 | L-Rhamnose degradation |
| ΔlacZ4787(::rrnB-3) | β-galactosidase |
| hsdR514 | EcoKI restriction- |
| | modification system subunit |

TABLE 7
Target Genes Identified in Selection Methods
in the presence of Furfural or Acetate

| | | | | Acetate | | Acetate | |
|---|---|---|---|---|---|---|---|
| Furfura | | Furfura | | | Acetate | | Acetate |
| Gene | Furf75_u | Gene | Furf75_d | Gene | MOPS_u | Gene | MOPS_d |
| csiD | 6.75 | ddpF | 8.36 | yegP | 12.84 | serS | 23.66 |
| talB | 6.65 | nagA | 7.21 | Tap | 9.86 | yahF | 12.77 |
| ydaL | 6.28 | ydiA | 7.05 | lacy | 6.44 | tqsA | 10.29 |
| yeeN | 6.15 | ycjN | 6.61 | ycjU | 5.43 | fabH | 8.94 |
| smg | 6.02 | rseB | 6.36 | | | aspS | 8.29 |
| | | | | | | yjdM | 8.16 |
| | | | | | | clcB | 7.87 |
| | | | | | | sdaA | 7.23 |
| | | | | | | ycbS | 7.16 |
| | | | | | | deoA | 7.01 |
| | | | | | | ynfM | 6.70 |
| | | | | | | rimL | 6.59 |
| | | | | | | Ydcl | 6.34 |
| | | | | | | Sra | 6.05 |
| | | | | | | ydiA | 5.75 |
| | | | | | | mcbR | 5.50 |
| | | | | | | ybeT | 5.43 |
| | | | | | | lpxT | 5.28 |
| | | | | | | ycjF | 5.21 |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A composition for increasing tolerance for furan-2-carbaldehyde (commonly known as furfural), and acetate by a microorganism, the microorganism having reduced lipopolysaccharide production compared to a control, comprising: a vector containing one or more genes including lpcA whose expression increases tolerance for furfural, and acetate by the microorganism compared to a control microorganism; and
a media.

2. The composition of claim 1, further comprising a bacterial culture.

3. The composition of claim 1, wherein the bacterial culture is E. coli.

4. The composition of claim 1, further comprising a vector containing other genes in the lipopolysaccharide (LPS) pathway that increase LPS production.

5. The composition of claim 1, further comprising an agent to inactivate araC in the microorganism.

6. The composition of claim 1, wherein increasing tolerance to furfural and acetate comprises increasing growth rate of the microorganism by 5% or more; or 15% or more; or 25% or more; or 35% or more; or over 100% or more relative to a control microorganism.

7. The composition of claim 1, wherein increasing tolerance to furfural and acetate comprises increasing tolerance to 0.5 g/L to 2.0 g/L of furfural and 2.5 to 30 g/L of acetate produced by the microorganism.

8. The composition of claim 1, wherein the vector comprises pEZseq (Lucigen) cloning systems, pSMART cloning systems (Lucigen), pACYCDuet cloning systems (Novagen) pBMT-1, pBMT-2, pBMT-3, pBMT-4, pBMT-5, pBMT-6, pBT-1, pBT-2, pBT-3, pBT-4, pBT-5, pBT-6, pBMTB-1, pBMTB-2, pBMTB-3, pBMTB-4, pBMTB-5, pBMTB-6, pBTB-1, pBTB-2, pBTB-3, pBTB-4, pBTB-5, pBTB-6, pBMTL-1, pBMTL-2, pBMTL-3, pBMTL-4, pBMTL-5 pBMTL-6, pBTL-1, pBTL-2, pBTL-3, pBTL-4, pBTL-5, pBTL-6 or any vector capable of inducing the one or more genes.

9. The composition of claim 1, wherein the vector stably integrates into the genome of the microorganism.

10. The composition of claim 1, further comprising supplementary amino acids.

11. A method for modulating tolerance to furfural and acetate by a microorganism comprising:
obtaining a vector associated with one or more genes including lpcA wherein expression of the one or more genes including lpcA modulates tolerance of at least furfural and acetate by the microorganism;
introducing the vector to the microorganism; and
expressing the one or more genes.

12. The method of claim 11, further comprising inactivating araC in the microorganism.

13. A modified bacterial organism for biofuels production comprising: a bacterial organism comprising an araC deletion or inactivation thereof wherein the modified bacterial organism has increased tolerance to byproducts of biomass hydrolysates than a control bacterial organism.

14. The modified bacterial organism of claim 13, further comprising increasing LPS production in the modified bacterial organism.

15. The modified bacterial of claim 13, wherein the bacterial organism is E. coli.

16. A modified bacterial organism comprising: a bacterial organism wherein lpcA expression is artificially upregulated by inducing overexpression or introducing additional lpcA gene copy numbers to the bacterial organism; and if present, deleting araC in the bacterial organism.

17. A kit comprising, an agent to delete araC in a microorganism; at least one container and a culture of microorganisms.

18. The kit of claim 17, wherein the culture of microorganisms is a bacterial culture.

19. The kit of claim 18, wherein the culture comprises Escherichia coli.
